(19) United States
(12) Patent Application Publication
(10) Pub. No.: US 2002/0142462 A1
(43) Pub. Date: Oct. 3, 2002

(54) Methods for mobilizing hematopoietic facilitating cells and hematopoietic stem cells into the peripheral blood

(76) Inventors: Suzanne T. Ildstad, Wynewood, PA (US); Tatiana D. Zorina, Aldan, PA (US)

Correspondence Address: Licata & Tyrrell P.C., 66 E. Main Street, Marlton, NJ, 08053, US

(21) Appl. No.: 10/078,328

(22) Filed: Feb. 15, 2002

Related U.S. Application Data
(63) Continuation of application No. 09/468,686, filed on Dec. 21, 1999, now abandoned.
Application No. 09/468,686 is a continuation of application No. 09/072,862, filed on May 5, 1998, now abandoned.
Application No. 09/072,862 is a continuation-in-part of application No. 08/986,511, filed on Dec. 8, 1997, now abandoned.
(60) Provisional application No. 60/066,821, filed on Nov. 26, 1997.

(51) Int. Cl. C12N 5/08; A61K 45/00

(52) U.S. Cl. 435/372; 424/93.7

(57) ABSTRACT

The present invention relates to methods for mobilizing hematopoietic facilitating cells (FC) and hematopoietic stem cells (HSC) into a subject's peripheral blood (PB). In particular, the invention relates to the activation of both FLT3 and granulocyte-colony stimulating factor (G-CSF) receptor to increase the numbers of FC and HSC in the PB of a donor. The donor's blood contains both mobilized FC and HSC, and can be processed and used to repopulate the destroyed lymphohematopoietic system of a recipient. Therefore, PB containing FC and HSC mobilized by the method of the invention is useful as a source of donor cells in bone marrow transplantation for the treatment of a variety of disorders, including cancer, anemia, autoimmunity and immunodeficiency. Alternatively, the donor's hematopoietic tissue, such as bone marrow, can be treated ex vivo to enrich selectively for FC and HSC populations by activating appropriate cell surface receptors.

This Application is a continuation-in-part of U.S. patent application Ser. No. 08/986,511, filed Dec. 8, 1997, which claims priority to United States Provisional Patent Application Serial No. 60/066,821, filed Nov. 26, 1997.

### 1. INTRODUCTION

The present invention relates to methods for mobilizing hematopoietic facilitating cells (FC) and hematopoietic stem cells (HSC) into a subject's peripheral blood (PB). In particular, the invention relates to the activation of both FLT3 and granulocyte-colony stimulating factor (G-CSF) receptor to increase the numbers of FC and HSC in the PB of a donor. The donor's blood contains both mobilized FC and HSC, and can be processed and used to repopulate the destroyed lymphohematopoietic system of a recipient. Therefore, PB containing FC and HSC mobilized by the method of the invention is useful as a source of donor cells in bone marrow transplantation for the treatment of a variety of disorders, including cancer, anemia, autoimmunity and immunodeficiency. Alternatively, the donor's hematopoietic tissue, such as bone marrow, can be treated ex vivo to enrich selectively for FC and HSC populations by activating appropriate cell surface receptors.

### 2. BACKGROUND OF THE INVENTION

2.1. Bone Marrow Transplantation

Bone marrow transplantation is a clinical procedure in which donor bone marrow cells are transplanted into a recipient for the reconstitution of the recipient's lymphohematopoietic system. Prior to the transplant, the recipient's own blood system is either naturally deficient or intentionally destroyed by agents such as irradiation. In cases where the recipient is a cancer patient, ablative-therapy is often used as a form of cancer treatment which also destroys the cells of the lymphohematopoietic system. Bone marrow transplantation is an effective form of treatment of hematologic tumors and anemias.

The success rate of bone marrow transplantation depends on a number of critical factors, which include matching between donor and recipient at the major histocompatibility complex (MHC) which encodes products that induce graft rejection, the enrichment of adequate numbers of hematopoietic progenitor cells in the donor cell preparation, the ability of such cells to durably engraft in a recipient and conditioning of the recipient prior to transplantation.

A serious impediment in bone marrow transplantation is the need for matching the MHC between donors and recipients through HLA tissue typing techniques. Matching at major loci within the MHC class I and class II genes is critical to the prevention of rejection responses by the recipient against the engrafted cells, and more importantly, donor cells may also mediate an immunological reaction to the host tissues referred to as graft-versus-host disease (GVHD). In order to facilitate graft acceptance by the host, immunosuppressive agents have often been employed, which render the patients susceptible to a wide range of opportunistic infections, and increases the risk of secondary malignancy development.

Tissue typing technology has ushered in dramatic advances in the use of allogeneic bone marrow cells as a form of therapy in patients with a spectrum of diseases, such as deficient or abnormal hematopoiesis, genetic disorders, enzyme deficiencies, hemoglobinopathies, autoimmune disorders, and malignancies. Conditioning of a recipient can be achieved by total body or total lymphoid irradiation. While methods to enrich for the HSC in a donor cell preparation have improved in recent years primarily due to the discovery of certain markers expressed by HSC such as CD34, it has been shown that highly purified HSC do not durably engraft in MHC-disparate recipients (El-Badri and Good, 1993, Proc. Natl. Acad. Sci. U.S.A., 90:9233; Kaufman et al., 1994, Blood, 84:2436). A second cell type referred to as FC is required for HSC to engraft. The FC display a phenotype of CD8⁺, CD3⁺, αβTCR⁻ and γδTCR⁻, and are capable of facilitating donor bone marrow cell engraftment in an allogeneic recipient (Kaufman et al., Blood, supra). The discovery of FC has made it possible to specifically deplete T cells from a donor cell preparation with the retention of FC and HSC for use in bone marrow transplantation to produce long-term donor cell engraftment and clinically controllable GVHD.

In view of the foregoing, the ability to enrich for both HSC and FC in a donor cell preparation, by either in vivo or ex vivo methods, is critical to the application of bone marrow transplantation as a form of therapy. Neoplastic transformation, immunodeficiency, genetic abnormalities, and even viral infections can all affect blood cells of different lineages and at different stages of development. Bone marrow transplantation provides a potential means for treating all such disorders. In addition, although bone marrow transplantation is not used as a direct form of treatment for solid tumors, it provides an important means of maintaining survival of patients following various ablative therapeutic regimens.

2.2. Mobilization of Peripheral Blood

Conventional bone marrow transplantation utilizes bone marrow cells harvested from the iliac crest of a donor.

This is a painful, invasive procedure which yields low numbers of the critical HSC and FC populations. The number of HSC naturally present in the bone marrow is extremely low and has been estimated to be on the order of about one per 10,000 to one per 100,000 cells (Boggs et al., 1982, J. Clin. Inv., 70:242 and Harrison et al., 1988, Proc. Natl. Acad. Sci. U.S.A., 85:822. Current methods of bone marrow transplantation strive to obtain at least 1 million CD34⁺ cells/kg of body weight for repopulating an ablated human bone narrow. This would require the infusion of about 10⁸ cells/kg. Furthermore, if the donor cell preparation contains contaminating tumor cells that must be purged prior to autologous re-infusion, the large number of total cells with a low percentage of CD34⁺ cells makes it technically difficult to perform adequate purging of tumor cells.

FC generally make up between about 0.5% and 8% of the cells found in physiological hematopoietic cell sources, and thus the concentration of FC in the PB is also relatively minute. The implantation of sufficient numbers of FC is critical to lymphohematopoietic repopulation of the recipient, and it appears that at least 0.8×10⁶ cells/kg are necessary for successful engraftment.

In an effort to obtain cells from a more convenient cell source than the bone marrow for use as donor cells, investigators have applied various hematopoietic growth factors to a donor to induce HSC into the PB in a process known as mobilization. Mobilization induces certain bone marrow cells to migrate into the circulating blood. The cells are then easily harvested by techniques well known in the art such as apheresis. Several growth factors or cytokines with hematopoietic activities have been used, including the interleukins (e.g., IL-7, IL-8 and IL-12), granulocyte-macrophage colony-stimulating factor (GM-CSF), granulocyte colony-stimulating factor (G-CSF), and steel factor (SLF) (Brasel, 1996, Blood 88:2004).

Certain cytokines involved in hematopoietic development function by activating receptor protein tyrosine kinases (pTKs). For example, the c-KIT pTK and its cognate ligand (KL) have been shown to play a role in hematopoiesis. Tyrosine kinases catalyze protein phosphorylation using tyrosine as a substrate for phosphorylation. Members of the tyrosine kinase family can be recognized by the presence of several conserved amino acid regions in the tyrosine kinase catalytic domain (Hanks et al., 1982, Science, 241:42-52).

A murine gene encoding a pTK which is expressed in cell populations enriched for stem cells and primitive uncommitted progenitors has been identified and is referred to as “fetal liver kinase-2” or “flk-2” by Matthews et al., 1991, in Cell, 65:1143-52. Rosnet et al. independently identified cDNA sequences from murine and human tissues relating to the same gene, which they named “flt3”, Genomics, 1991, 9:380-385, 1991, Oncogene, 6:1641-1650). The sequence for human flk2 is disclosed in WO 93/10136. Kuczynski et al. reported a gene known as “STK-1” which is the human homologue of murine flk2/flt3 (1993, Blood, 82(10):PA486).

The FLK2/FLT3 receptor is structurally related to subclass III PTKS such as α and β platelet-derived growth factor receptors (PDGF-R), colony-stimulating factor (CSF-1, also known as macrophage colony stimulating factor, M-CSF) receptor (C-FMS) and Steel factor (also known as mast cell growth factor, stem cell factor or kit ligand) receptor (c-KIT). The genes encoding these pTK appear to have major growth and/or differentiation functions in various cells, particularly in the hematopoietic system and in placental development (see Rosnet et al. in Genomics, supra).

A transmembrane ligand (FL) for the FLK2/FLT3 receptor was molecularly cloned (Lyman et al., 1993, Cell, 75:1157-1167). The protein was found to be similar in size and structure to the cytokines, M-CSF and SLF. FL promotes the growth of murine hematopoietic progenitor cells ex vivo and in vivo (Hudak et al., 1995, Blood 85:2747; Hirayama et al., 1995, Blood 85:1762; Brasel et al., 1996, Blood 88:2004).

Recent in vivo experiments with FL indicated that the administration of FL alone mobilized progenitor cells into the peripheral blood of mice (Brasel et al., 1996, Blood 88(6):2004). Subsequent experiments incorporated the use of G-CSF as a mobilization factor, and found increased mobilization of peripheral blood stem cells in murine models (Molineaux et al., 1997, Blood 89(11):3999; Brasel et al., 1997, Blood 90(9):3787). However, in some cases, the mobilized cells were unable to reconstitute lethally irradiated mice, possibly due to the dosage or scheduling of administration of the FL and the G-CSF.

The above-described mobilization techniques utilized FL and G-CSF in order to mobilize HSC. However, prior to the present invention, it was not known whether any technique could be used to mobilize FC into PB. Since it is known that HSC alone, or in a mixed bone narrow cell population, do not readily engraft in a recipient, and that PC are necessary for lymphohematopoietic reconstitution, there remains a need for a method which can mobilize both HSC and FC into the PB.

2.3. Ex vivo Enrichment of FC and HSC

As an alternative to in vivo mobilization of FC and HSC into a donor's peripheral blood, investigators have explored the possibility of enriching the HSC population ex vivo, by culturing a donor's hematopoietic tissue. However, prior to the present invention, it was not known whether any technique could be used to enrich FC in cell culture. As discussed in Section 2.2, supra, FC are necessary for lymphohematopoietic reconstitution. Thus, there remains a need for a method that can enrich both FC and HSC ex vivo.

### 3. SUMMARY OF THE INVENTION

The present invention relates to methods of mobilizing HSC and FC into the PB of a subject by stimulation of FLK2/FLT3 and G-CSF receptor, such that a high yield of HSC and FC can be retrieved and used for subsequent lymphohematopoietic reconstitution in a recipient. The present invention also relates to methods of enriching HSC and FC ex vivo in hematopoietic cell cultures by FLK2/FLT3 and G-CSF receptor stimulation.

The FL can be a mammalian FL, including a mouse or primate ligand, e.g., a human ligand. In other embodiments, the FL will be a recombinant FL; or will be administered through gene therapy; or will be administered in combination with an effective amount of a cytokine, sequentially or concurrently. Such cytokines include, but are not limited to, interleukins (IL) IL-1, IL-2, IL-3, IL-4, IL-5, IL-6, IL -7, IL-8, IL-9, IL-10 IL-11, IL-12, IL-13, IL-14, or IL-15, and a CSF, such as G-CSF, GM-CSF, M-CSF, or GM-CSF/IL-3 fusions, as well as other growth factors such as CSF-1, SCF, SF, EPO, leukemia inhibitory factor (LIF), or fibroblast growth factor (FGF), as well as C-KIT ligand, and TNF-α.

For in vivo mobilization, the route of administration of FL and G-CSF can be parenteral, topical, intravenous, intramuscular, intradermal, subcutaneous, or in a slow release formulation or device. Peripheral blood mononuclear cells (PBMC) are collected from the donor, preferably when the FC and the HSC reach peak levels in the circulation. The optimal timing for collection will vary depending upon the dosage, timing, and mode of administration of the cytokines.

Alternatively, the donor's hematopoietic tissue, including but not limited to bone marrow and blood, can be collected by methods well known to those of skill in the art, and treated ex vivo to activate the TNF receptor, the GM-CSF receptor, the G-CSF receptor, the SCF receptor, the IL-7 receptor, the IL-12 receptor, or FLT3.

In another embodiment, the invention provides a composition comprising an effective combination of FL and G-CSF. The composition will often further comprise a pharmaceutically acceptable carrier.

The invention is based, in part, on Applicants' discovery that the HSC and FC fractions in the peripheral blood of animals treated with factors that stimulate FLT3 and the G-CSF receptor is significantly higher than in untreated animals, as well as the discovery that stimulation of the SCF, TNF and GM-CSF receptors or FLT3 ex vivo selectively enriches HSC and FC populations.

### 4. BRIEF DESCRIPTION OF THE DRAWINGS

FIG. 1A and B FC are defined as CD8⁺ but αβTCR⁻ and γδTCR⁻.

FIG. 2 The number of white blood cells in peripheral blood was most effectively increased by the combination of G-CSF and FL.

FIG. 3A Administration of FL and G-CSF mobilized the highest number of HSC into the PB as compared to FL alone, G-CSF alone and saline. Peak levels were achieved on day 10.

FIG. 3B Administration of FL and G-CSF mobilized the highest number of FC into the PB as compared to FL alone, G-CSF alone and saline. Peak levels were achieved on day 10.

FIGS. 4A-C: Kinetics of mobilization of (A) peripheral blood mononuclear cells (PBMC), (B) HSC, and (C) FC under treatment with FL alone (▴), G-CSF alone (⋄), and FL plus G-cSF (□) or carrier ( - - - ). FL (10 μg/mouse) was injected subcutaneously for 10 days and G-CSF (7.5 μg/mouse) from day 4 to 10. (A) PB was obtained daily and PBMC were counted. The percentage of HSC (lineage⁻/SCA-1⁺/c-kit⁺) and FC (CD8⁺/αβTCR⁻/γδTCR⁻) was analyzed by flow cytometry and absolute numbers of HSC and FC were calculated based on percentage of total and individual PBMC counts. Results represent the mean (SEM) of two different experiments (n=5 per group). PBMC or absolute numbers of FC and HSC that differed significantly from controls are marked (* p<0.005 or ** p<0.0005).

FIGS. 5A-C: Percentage of HSC, FC, and CD8⁺ T-cells in PB under treatment with (A) FL alone, (B) G-CSF alone or (C) FL plus G-CSF. FL (10 μg/mouse) was injected subcutaneously from day 1 to 10 and G-CSF (7.5 μg/mouse) from day 4 to 10. PB was stained for HSC (lineage⁻/SCA-1⁺/c-kit⁺) (▪) and FC (CD8⁺/αβTCR⁻/γδTCR⁻) (□) and CD8⁺ T-cells (CD8⁺/αβTCR⁺) ( [symbol] ). Results show the mean (SEM) percentage before and on day 7 and day 10 of growth factor administration. Percentages of HSC, FC, or CD8⁺ T-cells that differed significantly from day 0 values are marked (* p<0.05; ** p<0.005 or *** p<0.0005).

FIGS. 6A-F: Distribution of HSC, FC, and CD8⁺T-cells in (A-C) bone marrow and (D-F) spleen of B10.BR mice treated with FL alone (10 μg/mouse; day 1 to 10), G-CSF alone (7.5 μg/mouse; day 4 to 10) or FL plus G-CSF. Animals were euthanized before, on day 7 or on day 10 of GF administration.

Long bones and spleens were harvested and processed for each individual animal. Bone marrow cells and splenocytes were analyzed for the percentage of HSC (lineage /SCA-1⁺/c-kit⁺) (▪), FC (CD8⁺/αβTCR⁻/γδTCR⁻) (□), and CD8⁺ T-cells (CD8⁺/αβTCR⁺) ( [symbol] ) by flow cytometry. Results represent the mean (SEM) percentage on total bone marrow and total splenocytes. Percentages of HSC and FC that differed significantly from day 0 values are marked (* p<0.05 or ** p<0.005).

FIGS. 7A-D: Survival (30 days) of lethally irradiated recipients (C57BL/10 SnJ) transplanted with mobilized PB from donor mice (B10.BR). Donors were treated once daily with FL alone (▴) (10 μg/mouse; day 1 to 10), C-CSF alone (⋄) (7.5 μg/mouse; day 4 to 10), FL plus G-CSF (□), or carrier only (●). PBMC were obtained from donors after 7 days (A and B) or 10 days (C and D) of GF administration and pooled for each group. Recipients were injected IV with 1×10⁶ or 2.5×10⁶ PBMC 3 to 5 hours after irradiation (4 to 7 mice per group). There was a significantly greater survival of mice reconstituted with PBMC from FL and FL plus G-CSF treated donors when compared to G-CSF mobilized PBMC (see results) or control animals.

FIGS. 8A-F: Flow cytometric analysis of PB obtained from a representative chimera 30 days after reconstitution with mobilized PB. C57BL/10SnJ mice (H-2K^{b}) were lethally irradiated and transplanted with varying numbers of PBMC from growth factor treated B10.BR donors (H-2K^{k}). PB from unmanipulated C57BL/10SnJ and B10.BR mice served as controls. Lineage derivation of PBMC was analyzed based on forward and side scatter and the percentage of cells residing in a lymphocyte (R1), monocyte (R2) or granulocyte gate (R3) was calculated. (A) The majority of PBMC in engrafted recipients were located in the granulocyte gate, (B and C) while most of PBMC from untreated controls resided in the lymphocyte gate. (D-F) In addition PB was stained with mAb specific for recipient (H-2K^{b}) and donor (H-2k^{k}) MHC class I and gated populations were analyzed by two-color flow cytometry. (D) Gated lymphocytes from engrafted recipient expressed exclusively donor MHC class I. Positive staining for donor but negative staining for recipient MHC class I was also observed when gated granulocytes and monocytes were analyzed (data not shown).

FIG. 9: Long-term survival (>6 months) of lethally irradiated and transplanted recipients was calculated using Kaplan-Meier estimates. B10 mice received 1×10⁶ to 5×10⁶ PBMC from B10.BR donors treated with FL alone, G-CSF alone or FL+G-CSF (n≧6 per group). Controls were transplanted with similar numbers of PBMC from untreated donors or 1×10⁶ bone marrow cells. Survival between different groups were compared using Wilcoxon test and significant differences are marked (*p<0.0001). The follow up ranged from 3 to 6 months.

FIG. 10: Assessment of longer term engraftment of mobilized HSC and FC by three-color flow cytometry. PB was obtained from lethally irradiated C57BL/10SnJ MICE (h-2K^{b}) 6 months after reconstitution with PBMC from GF-treated B10.BR mice (H -2K^{k}) and stained with lineage- and donor-specific mabs. Unmanipulated C57BL/10SnJ and B10.BR mice served as controls (data not shown).

Figure shows results of a representative long term surviving chimera. (A) Lymphocytes (R1) and granulocytes/macrophages (R2) were gated based on forward and side scatter. Engraftment of multiple donor derived cell lines including (B) B-cells. (C) T-cells, (D) NK cells, (E) granulocytes and (F) macrophages were detectable 6 months after transplantation indicating HSC engraftment.

FIGS. 11A and B Cultured cells facilitate allogeneic stem cell engraftment.

FIGS. 12A and B FC cell markers on cells generated in culture.

FIGS. 12C and D Lymphoid Dendritic cell (LDC) markers on cells generated in culture.

### 5. DETAILED DESCRIPTION OF THE INVENTION

The present invention relates to methods of mobilizing HSC and FC, methods of enriching HSC and FC, and uses of the cells for lymphohematopoietic repopulation of a recipient. For clarity of discussion, the specific procedures and methods described herein are exemplified using a murine model; they are merely illustrative for the practice of the invention. Analogous procedures and techniques are equally applicable to all mammalian species, including human subjects, in terms of mobilization of PB and the subsequent use of the HSC and FC from a donor for transplantation to a human recipient. Therefore, human HSC and FC having a similar phenotype and function may be used under the conditions described herein. Further, non-human animal HSC and FC may also be used to enhance engraftment of xenogeneic cells in human patients.

5.1. Donor Cells for Bone Marrow Transplantation

The HSC and FC are two major cell types necessary for successful repopulation of a destroyed or deficient lymphohematopoietic system. These cells are readily identified and enriched in a mixed cell population based on their unique profiles of phenotypic markers. Antibodies specific for these markers are commercially available, and can be used in combination to determine the presence of HSC and FC in PB. In order to obtain a purified population of HSC and FC from a cell mixture, positive and negative selection procedures may be employed. In addition, other cell separation methods such as density gradient centrifugation and elutriation may be used.

5.1.1. Hematopoietic Facilitating Cells

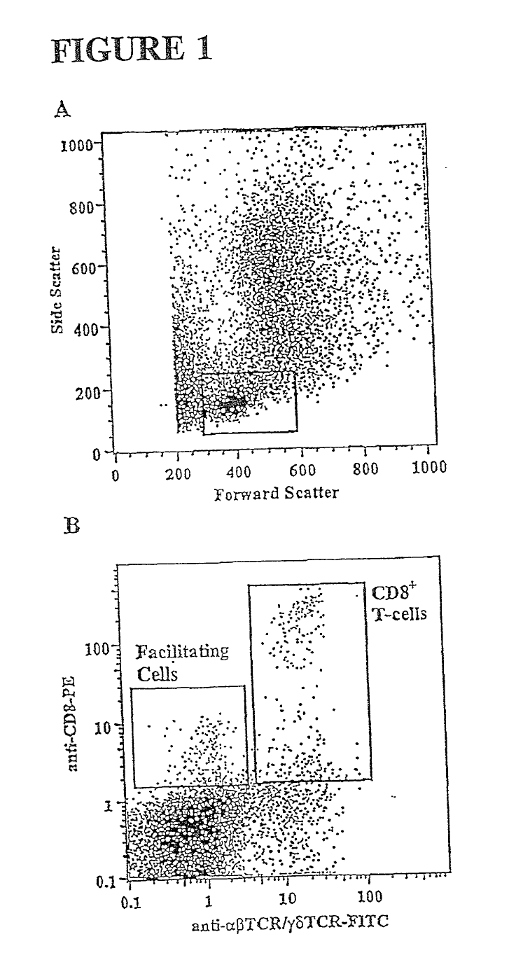

FC display a phenotype of CD8⁺, αβ-TCR⁻, and γδ-TCR⁻, which distinguishes them from T cells. In addition, the phenotype of a FC is further characterized as CD4⁻, CD5⁺, CD16⁻, CD19⁻, CD20⁻, CD56⁻, mature myeloid lineage (CD14⁻), Class II⁺, CD45⁺, CD45R⁺ and, THY1⁺ (FIG. 1). Although the Applicants' own work supports the CD3⁺ phenotypic characterization of the hematopoietic facilitatory cell population, recent work of other groups raises the possibility that these cells may, in fact, be CD3⁻. See, e.g., Aguila H. et al., Immunological Rev., 1997, 157:13-36. However, the hematopoietic facilitatory cells are readily identifiable by the other cell surface markers listed above. Morphologically, purified FC are distinct from all other hematopoietic cell types, including lymphocytes Furthermore, these cells function in a MHC-specific fashion in that optimal engraftment of bone marrow cells is achieved if they are of the same MHC haplotype as the FC. The FC can also facilitate xenogeneic bone marrow engraftment across species barriers in establishing mixed lymphohematopoietic chimerism.

When co-administered with other bone marrow cells, especially the HSC, the FC enhance their engraftment, without apparent adverse biologic activities. In fact, the ability of the FC to enhance the engraftment of bone marrow cells in establishing lymphohematopoietic chimerism without producing GVHD also induces donor-specific tolerance to permit the permanent acceptance of donor's cells, tissues and organs.

It is possible that particular species or certain strains of particular species possess FC which are also capable of facilitating engraftment of stem cells and other bone marrow components which are not MHC-specific. Furthermore, FC and HSC may not need to be matched at their MHC entirely. Since there are subregions within both Class I and Class II genes of the MHC, matching at only one of these regions may be sufficient for the FC to enhance stem cell engraftment.

5.1.2. Hematopoietic Stem Cells

Human HSC reside in the CD34⁺ fraction, although not all CD34⁺ cells are capable of giving rise to various mature blood lineages. More particularly, U.S. Pat. No. 5,061,620 characterizes bone marrow stem cells as CD34⁺, CD3⁻, CD7⁻, CD8⁻, CD10⁻, CD14⁻, CD15⁻, CD19⁻, CD20⁻, CD33⁻, and THY1⁺ (or low level expression). The phenotype of CD3⁻, CD8⁻, CD10⁻, C19⁻, CD20⁻, and CD33⁻ is referred to as “lineage⁻” (European Patent No. 451,611). Moreover, HSC are believed to be Class II⁺. Because a homologous CD34 marker had not been identified for rodent stem cells until recently, the phenotype of HSC in murine models is generally characterized as c-KIT⁺, SCA⁺, and lineages. Such murine HSC are considered in the art to be the equivalent to the CD34⁺ human HSC.

5.2. Mobilization of Cells into the Peripheral Blood

5.2.1. FL and G-CSF

The present invention provides a method for mobilizing HSC and FC into the peripheral blood of a subject. This can generally be achieved by treatment of a donor with agents that stimulate the FLK2/FLT3 protein and G-CSF receptor expressed by hematopoietic cells. Alternatively, stimulation of the FLK2/FLT3 protein alone may be sufficient to mobilize FC and HSC in some instances.

In a particular embodiment illustrated by working examples, FL and G-CSF were used in combination to mobilize HSC and FC into PB. The term “FL” as used herein encompasses proteins such as those described in U.S. Pat. No. 5,554,512 to Lyman et al., as well as proteins having a high degree of structural similarity that bind to FLT3 resulting in activation of pTK. FL includes membrane-bound proteins, soluble or truncated proteins which comprise primarily the extracellular portion of the protein and antibodies or biologically active fragments that bind FLT3 (U.S. Pat. No. 5,635,388). The term “G-CSF” encompasses any ligands, including agonistic antibodies, that activate the G-CSF receptor.

Polynucleotides encoding FL and G-CSF have been described, e.g., Hannum et al., (1994) Nature 368:643-648; Lyman et al., (1994) Blood 83:2795-2801; and Lyman et al., (1993) Cell 75:1157-1167. See also U.S. Pat. No. 5,554,512 to Lyman et al., WO 94/26891 to Hannum et al., and WO 96/34620 to Hudak and Rennick. Descriptions of vectors useful for expression are well known to those skilled in the art, and are included in Pouwels et al., (1985 and Supplements) Cloning Vectors: A Laboratory Manual, Elsevier, N.Y.; Rodriguez et al., (1988) (eds.); Vectors: A Survey of Molecular Cloning Vectors and Their Uses, Buttersworth, Boston, Chapter 10, pp. 205-236; Okayama et al., (1985) Mol. Cell Biol. 5:1136-1142; Thomas et al., (1987) Cell 51:503-512; Low, (1989) Biochim. Bionhys. Acta 988:427-454; Tse et al., (1985) Science 230:1003-1008; and Brunner et al., (1981) J. Cell Biol. 114:1275-1283.

Encompassed within the present invention are also variants which are proteins or peptides having substantial amino acid sequence homology with the amino acid sequence of FL which bind to FLT3. Techniques for producing such variants are well known, and descriptions of how comparisons are made can be found, e.g., in Needleham et al., (1970) J. Mol. Biol. 48:443-453; Sankoff et al., (1983) Chapter One in Time Warps, String Edits, and Macromolecules: The Theory and Practice of Sequence Comparison, Addison-Wesley, Reading, Mass.; and software packages from IntelliGenetics, Mountain View, Calif.; the University of Wisconsin Genetics Computer Group, Madison, Wis. Methods to manipulate nucleic acids are described, e.g., in Sambrook et al., (1989) Molecular Cloning: A Laboratory Manual (2d ed.), Vols. 1-3, Cold Spring Harbor Laboratory; Ausubel et al., Biology, Greene Publishing Associates, Brooklyn, N.Y.; Ausubel et al., (1987 and Supplements) Current Protocols in Molecular Biology, Greene/Wiley, New York; Innis et al., (eds.) (1980); Cunningham et al., (1989) Science 243:1330-1336; O'Dowd et al., (1988) J. Biol. Chem. 263:15985-15992; and Beaucage and Carruthers, (1981) Tetra. Letts. 22:1859-1862.

FL and G-CSF may be prepared by chemical synthetic methods as described in U.S. Pat. No. 5,554,512 to Lyman et al., WO 94/26891 to Hannum et al., and WO 96/34620 to Hudak and Rennickin. General descriptions of synthetic peptide synthesis are found, e.g., in Merrifield, (1963) J. Amer. Chem. Soc. 85:2149-2156; Merrifield, (1986) Science 232:341-347; Atherton et al., (1989) Solid Phase Peptide Synthesis: A Practical Approach, IRL Press, Oxford; Stewart and Young, (1984) Solid Phase Peptide Synthesis, Pierce Chemical Co., Rockford, Ill.; Bodanszky and Bodanszky, (1984) The Practice of Peptide Synthesis, Springer-Verlag, New York; and Bodanszky, (1984) The Principles of Peptide Synthesis, Springer-Verlag, New York.

Recombinantly or synthetically prepared ligand and fragments thereof can be isolated and purified by peptide separation methods, e.g., by extraction, precipitation, electrophoresis, and various forms of chromatography, and the like. FL and G-CSF can be obtained in varying degrees of purity depending upon its desired use.

The present invention is not limited to ligands which interact with the extracellular domains of the FLT3 protein and the G-CSF receptor. Current pharmaceutical research is aimed at identifying small organic molecules that gain access to a cell and interact with the intracellular catalytic domain of transmembrane proteins, or with downstream components of the signal transduction pathway, to obtain an effect similar to receptor ligand binding. The present invention contemplates the use of such small organic molecules, hereinafter referred to as “activation agents”, to mobilize FC and HSC into the peripheral blood.

5.2.2. Administration of FL and G-CSF

The FL and G-CSF or the activation agents are purified and suspended in an appropriate solution for in vivo administration. The reagents can be combined for therapeutic use with additional active or inert ingredients, e.g., in conventional pharmaceutically acceptable carriers or diluents, e.g., with physiologically innocuous stabilizers and excipients. These combinations can be sterile filtered and placed into dosage forms as by lyophilization in dosage vials or storage in stabilized aqueous preparations.

The quantities of reagents necessary for effective therapy will depend upon many different factors, including means of administration, target site, physiological state of the patient, and other medicants administered. Thus, treatment dosages should be titrated to optimize safety and efficacy. The FL will often be administered to the donor at a dose of between 50 μg/kg and 500 μg/kg. The G-CSF will typically be administered to the donor at a dose of between 25 μg/kg and 500 μg/kg. Typically, dosages used ex vivo may provide useful guidance in the amounts useful for in situ administration of these reagents. Animal testing of effective doses for treatment of particular disorders will provide further predictive indication of human dosage. Various considerations are described, e.g., in Gilman et al., (eds.) (1990) Goodman and Gilman's: The Pharmacological Bases of Therapeutics, 8th Ed., Pergamon Press, and Remington's Pharmaceutical Sciences, 17th ed. (1990), Mack Publishing Co., Easton, Pa. Methods for administration are discussed therein, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Dosage ranges for FL and G-CSF would ordinarily be expected to be in amounts of at least about lower than 1 Mm concentrations, typically less than about 10 μM concentrations, usually less than about 100 Nm, preferably less than about 10 Pm (picomolar), and most preferably less than about 1 Fm (femtomolar), with an appropriate carrier. Slow release formulations, or a slow release apparatus will often be utilized for continuous administration.

The FL and G-CSF or the activation agents may be administered directly to a subject or it may be desirable to conjugate it to carrier proteins such as ovalbumin or serum albumin prior to administration. While it is possible for the active ingredient to be administered alone, it is preferable to present it as a pharmaceutical formulation. Formulations typically comprise at least one active ingredient, together with one or more acceptable carriers thereof. Each carrier should be both pharmaceutically and physiologically acceptable in the sense of being compatible with the other ingredients and not injurious to the patient. Formulations include those suitable for oral, rectal, nasal, topical, or parenteral (including subcutaneous, intramuscular, intravenous and intradermal) administration. The formulations may conveniently be presented in unit dosage form and may be prepared by many methods well known in the art of pharmacy. See, e.g., Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds.) (1993) Pharmaceutical Dosage Forms, Parenteral Medications Dekker, N.Y.; Lieberman, et al. (eds.) (1990) Pharmaceutical Dosage Forms: Tablets Dekker, N.Y.; and Lieberman, et al. (eds.) (1990) Pharmaceutical Dosage Forms: Disperse Systems Dekker, N.Y. The methods of the invention may be combined with or used in association with other therapeutic agents.

In particular, the administration will likely be in combination with other aspects in a therapeutic course of treatment. In particular, the administration may involve multiple administrations, in combination with other agents, e.g., (IL) IL-1, IL-2, IL-3, IL-4, IL-5, IL-6, IL-7, IL-8, IL-9, IL-10 IL-11, IL-12, IL-13, IL-14, or IL-15, GM-CSF, M-CSF, or GM-CSF/IL-3 fusions, or other growth factors such as CSF-1, SF, EPO, leukemia inhibitory factor (LIF), or fibroblast growth factor (FGF), as well as C-KIT ligand, and TNF-α.

Blood containing mobilized HSC and FC may be collected from the donor by means well known in the art, preferably by apheresis. In order to ensure capture of a repopulating quantity of cells, it is preferable to collect the donor's blood when the levels of mobilized FC and HSC peak. The dosage, timing, and route of cytokine administration are likely to affect the kinetics of FC and HSC mobilization, such that peak levels of FC and HSC may be obtained on different days following different cytokine administration protocols. In order to optimize the number of FC and HSC harvested from mobilized blood, the levels of FC and HSC can be monitored by methods well known to those of skill in the art, and collection timed to coincide with FC and HSC peaks.

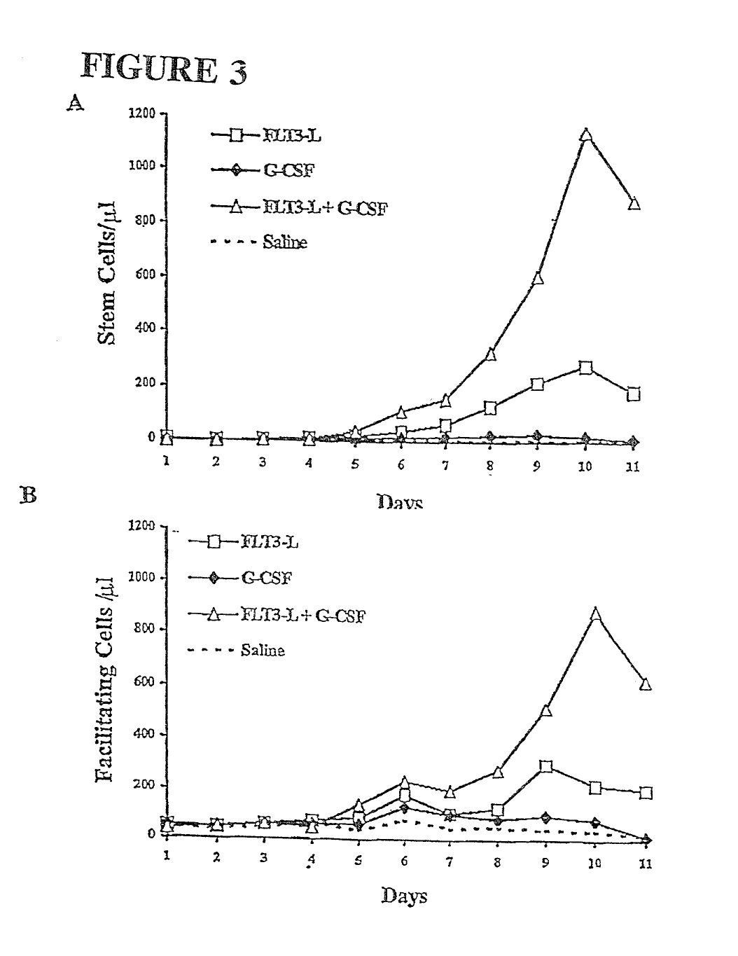

For example, when FL and G-CSF are administered together by subcutaneous injection, as in the method of Examples 1 and 2, infra, the levels of both FC and HSC appear to peak 8 to 10 days after initiation of cytokine treatment (see FIGS. 3A and 3B). Those skilled in the art can easily determine when FC and HSC levels have peaked following different cytokine administration protocols, and harvest the FC and HSC from the donor's peripheral blood at that time.

While it is preferred to treat the donor with both FL and G-CSF, in another embodiment, FL may be administered alone, and the donor's blood collected between days 5 and 7 of cytokine administration, when the FC and HSC levels peak, or between days 8 and 11 of cytokine administration, when they appear to peak again (See FIGS. 3A and 3B).

It should be noted that peak mobilization of FC and HSC may not coincide exactly, so that collection at the peak of FC mobilization may result in collection of HSC at a sub-peak level, and vice versa. Although collection at peak levels of mobilization of both FC and HSC is preferred, collection may also be made when the mobilization level of one cell population is peak and the other sub-peak, or when both are sub-peak. For example, when FL is administered alone by subcutaneous injection, as in the method of Example 1, infra, the level of FC appears to peak on day 10, while the level of HSC appears to peak on day 9, such that collection on day 10 would capture peak levels of FC and sub-peak levels of HSC, while collection on day 11 would capture sub-peak levels of both (See FIGS. 3A and 3B).

5.3. Enrichment of Donor Cells ex vivo

In another embodiment, the present invention provides a method for enriching HSC and FC ex vivo by treating any cell source in which they reside with factors that stimulate the TNF and GM-CSF receptors. Alternatively, in view of the in vivo mobilization results, factors that stimulate FLT3 and the G-CSF receptor, such as FL and G-CSF, may also be used. More particularly, hematopoietic tissues such as bone marrow and blood can be harvested from a donor by methods well known to those skilled in the art, and treated with TNFα, GM-CSF, FL, SCF, IL-7, IL-12, and G-CSF, either singularly or in combination, to enrich selectively for PC and HSC. Prior to harvesting the hematopoietic tissue, the donor may be treated with cytokines to increase the yield of hematopoietic cells, such as TNFα, GM-CSF, FL, and G-CSF, but no pre-treatment is required. At a minimum, the starting cell population must contain FC and HSC.

The cells harvested from the donor are cultured ex vivo for several days in medium supplemented with TNFα, GM-CSF, FL, SCF, IL-7, IL-12, and G-CSF, either singularly or in combination. The concentration of GM-CSF administered would typically be in the range of 1,000 U/ml. In an alternative embodiment, TNFα may also be administered, typically at a concentration of 200 U/ml. Appropriate concentrations of G-CSF, SCF, IL-7, IL-12, and FL can be readily determined by those of skill in the art, as by titration experiments or by reference to the working examples provided herein.

In some applications, it may be desirable to treat the cultured cells to remove GVHD causing cells, using the methods described for mobilized blood in Section 5.4, infra. The enriched FC and HSC may then be selectively collected from the culture using techniques known to those of skill in the art, such as those described in section 5.4, infra.

In order to ensure enrichment of FC and HSC to a repopulating quantity, it is preferable to collect the cultured cells when the levels of FC and HSC peak. As with in vivo mobilization, ex vivo enrichment of cultured hematopoietic cells produces peak levels of FC and HSC on different days depending on the cytokine administration protocol used. In order to optimize the number of FC and HSC collected from cultured cells, the levels of FC and HSC can be monitored by methods well known to those of skill in the art, and collection timed to coincide with FC and HSC peaks.

It should again be noted that the method of the invention encompasses collection at a time when one cell population has been enriched to peak levels, and the other is sub-peak, or when both are sub-peak.

Following collection, the FC and HSC can be resuspended and administered to the recipient in the manner and quantity described for administration of mobilized FC and HSC in Section 5.5, infra.

5.4. Preparation of Donor Cells from Mobilized Blood or Bone Marrow

Once the Hsc and FC have been mobilized into a subject's PB or enriched in the cultured cells, they may be used as donor cells in the form of total white blood cells or peripheral blood mononuclear cells, or selectively enriched by various methods which utilize specific antibodies which preferably bind specific markers to select those cells possessing or lacking various markers. These techniques may include, for example, flow cytometry using a fluorescence activated cell sorter (FACS) and specific fluorochromes, biotin-avidin or biotin-streptavidin separations using biotin conjugated to cell surface marker-specific antibodies and avidin or streptavidin bound to a solid support such as affinity column matrix or plastic surfaces, magnetic separations using antibody-coated magnetic beads, destructive separations such as antibody and complement or antibody bound to cytotoxins or radioactive isotopes.

If the mobilized blood is used for an autologous transplant, the peripheral blood mononuclear cells (PBMC) may be re-infused into the patient without modifications, with the exception that in the case of a cancer patient, the cell preparation is first purged of tumor cells. In contrast, if the mobilized blood is transferred into an allogeneic or xenogeneic recipient, the PBMC may first be depleted of GHVD-producing cells, leaving the HSC and FC enriched in the PBMC population. In that connection, the PBMC may be treated with anti-αβTCR and anti-γδTCR antibodies to deplete T cells, anti-CD19 to deplete B cells and anti-CD56 to deplete NK cells. It is important to note that anti-CD8,-CD3,-CD2, and -Thy-1 antibodies should not be used to deplete GVHD producing cells. The use of anti-CDS, anti-CD3 and anti-CD2 antibodies would deplete both T cells and FC, and an anti-Thy1 antibody would deplete T cells, FC and HSC. Therefore, it is important to choose carefully the appropriate markers as targets for selecting the cells of interest and removing undesirable cell types.

Separation via antibodies for specific markers may be by negative or positive selection procedures. In negative separation, antibodies are used which are specific for markers present on undesired cells. Cells bound by an antibody may be removed or lysed and the remaining desired mixture retained. In positive separation, antibodies specific for markers present on the desired cells are used. Cells bound by the antibody are separated and retained. It will be understood that positive and negative separations may be used substantially simultaneously or in a sequential manner. It will also be understood that the present invention encompasses any separation technique which can isolate cells based on the characteristic phenotype of the HSC and FC as disclosed herein.

The most common technique for antibody based separation has been the use of flow cytometry such as by a FACS. Typically, separation by flow cytometry is performed as follows. The suspended mixture of hematopoietic cells are centrifuged and resuspended in media. Antibodies which are conjugated to fluorochrome are added to allow the binding of the antibodies to specific cell surface markers. The cell mixture is then washed by one or more centrifugation and resuspension steps. The mixture is run through a FACS which separates the cells based on different fluorescence characteristics. FACS systems are available in varying levels of performance and ability, including multi-color analysis. The FC and HSC can be identified by a characteristic profile of forward and side scatter which is influenced by size and granularity, as well as by positive and/or negative expression of certain cell surface markers.

Other separation techniques besides flow cytometry may provide for faster separations. One such method is biotin-avidin based separation by affinity chromatography. Typically, such a technique is performed by incubating the washed bone marrow with biotin-coupled antibodies to specific markers followed by passage through an avidin column. Biotin-antibody-cell complexes bind to the column via the biotin-avidin interaction, while other cells pass through the column. Finally, the column-bound cells may be released by perturbation or other methods. The specificity of the biotin-avidin system is well suited for rapid positive separation.

Flow cytometry and biotin-avidin techniques provide highly specific means of cell separation. If desired, a separation may be initiated by less specific techniques which, however, can remove a large proportion of non-HSC and non-FC from the hematopoietic cell source. It is generally desirable to lyse red blood cells from mobilized blood before use. For example, magnetic bead separations may be used to initially remove lineage committed, differentiated hematopoietic cell populations, including T-cells, B-cells, natural killer (NK) cells, and macrophages (MAC), as well as minor cell populations including megakaryocytes, mast cells, eosinophils, and basophils. Desirably, at least about 70% and usually at least about 80% of the total hematopoietic cells present can be removed.

A preferred initial separation technique is density-gradient separation. Here, the mobilized blood is centrifuged and the supernatant removed. The cells are resuspended in, for example, RPMI 1640 medium (Gibco) with 10% HSA and placed in a density gradient prepared with, for example, Ficoll or Percoll or Eurocollins media. The separation may then be performed by centrifugation or may be performed automatically with, for example, a Cobel & Cell Separator '2991 (Cobev, Lakewood, Colo.). Additional separation procedures may be desirable depending on the source of the hematopoietic cell mixture and on its content.

Although separations based on specific markers are disclosed, it will be understood that the present invention encompasses any separation based on the characterization of the HSC and FC disclosed herein which will result in a cellular composition comprising a high concentration of HSC and FC, whether that separation is a negative separation, a positive separation, or a combination of negative and positive separations, and whether that separation uses cell sorting or some other technique, such as, for example, antibody plus complement treatment, column separations, panning, biotin-avidin technology, density gradient centrifugation, or other techniques known to those skilled in the art. It will be appreciated that the present invention encompasses these separations used on any mammal including, but not limited to humans, nonhuman primates, rats, mice, and other rodents.

5.5. Uses of Mobilized Blood and Enriched Cultured Cells as Donor Cells

The HSC and FC contained in enriched cell cultures or mobilized blood may be used in the form of total mononuclear cells, or partially purified or highly purified cell populations. If these cellular compositions are separate compositions, they are preferably administered simultaneously, but may be administered separately within a relatively close period of time. The mode of administration is preferably but not limited to intravenous injection.

Once administered, it is believed that the cells home to various hematopoietic cell sites in the recipient's body, including bone marrow. The number of cells which should be administered is calculated for a specific species of recipient. For example, in rats, the T-cell depleted bone marrow component administered is typically between about 1×10⁷ cells and 5×10⁷ cells per recipient. In mice, the T-cell depleted bone marrow component administered is typically between about 1×10⁶ cells and 5×10⁶ cells per recipient. In humans, the T-cell depleted bone marrow component administered is typically between about 1×10⁸ cells and 3×10⁸ cells per kilogram body weight of recipient. For cross-species engraftment, larger numbers of cells may be required.

In mice, the number of purified FC administered is preferably between about 1×10⁴ and 4×10⁵ FC per recipient. In rats, the number of purified FC administered is preferably between about 1×10⁶ and 30×10⁶ FC per recipient. In humans, the number of purified FC administered is preferably between about 5×10⁴ and 10×10⁶ FC per kilogram recipient.

In mice, the number of HSC administered is preferably between about 100 and 300 HSC per recipient. In rats, the number of HSC administered is preferably between about 600 and 1200 HSC per recipient. In humans, the number of HSC administered is preferably between about 1×10⁵ and 1×10⁶ HSC per recipient. The amount of the specific cells used will depend on many factors, including the condition of the recipient's health. In addition, co-administration of cells with various cytokines may further promote engraftment.

In addition to total body irradiation, a recipient may be conditioned by immunosuppression and cytoreduction by the same techniques as are employed in substantially destroying a recipient's immune system, including, for example, irradiation, toxins, antibodies bound to toxins or radioactive isotopes, or some combination of these techniques. However, the level or amount of agents used is substantially smaller when immunosuppressing and cytoreducing than when substantially destroying the immune system. For example, substantially destroying a recipient's remaining immune system often involves lethally irradiating the recipient with 950 rads (R) of total body irradiation (TBI). This level of radiation is fairly constant no matter the species of the recipient. Consistent xenogeneic (rat→mouse) chimerism has been achieved with 750 R TBI and consistent allogeneic (mouse) chimerism with 600R TBT. Chimerism was established by PB typing and tolerance confirmed by mixed lymphocyte reactions (MLR) and cytotoxic lymphocyte (CTL) response.

The mobilized blood and enriched cultured cells prepared in accordance with the present invention may be used for establishing both allogeneic chimerism and xenogeneic chimerism. Xenogeneic chimerism may be established when the donor and recipient as recited above are different species. Xenogeneic chimerism between rats and mice, between hamsters and mice, and between chimpanzees and baboons has been established. Xenogeneic chimerism between humans and other primates is also possible. Xenogeneic chimerism between humans and other mammals, such as pig, is equally viable.

It will be appreciated that, though the methods disclosed above involve one recipient and one donor, the present invention encompasses methods such as those disclosed in which HSC and purified FC from two donors are engrafted in a single recipient.

It will be appreciated that the mobilized cells and enriched cultured cells of the present invention are useful in reestablishing a recipient's hematopoietic system by substantially destroying the recipient's immune system or immunosuppressing and cytoreducing the recipient's immune system, and then administering to the recipient syngeneic or autologous cell compositions comprising syngeneic or autologous purified FC and HSC which are MHC-identical to the FC.

The ability to establish successful allogeneic or xenogeneic chimerism allows for vastly improved survival of transplants. The present invention provides for methods of transplanting a donor physiological component, such as, for example, organs, tissue, or cells. Examples of successful transplants in and between rats and mice using these methods include, for example, islet cells, skin, hearts, livers, thyroid glands, parathyroid glands, adrenal cortex, adrenal medullas, and thymus glands. The recipient's chimeric immune system is completely tolerant of the donor organ, tissue, or cells, but competently rejects third party grafts. Also, bone marrow transplantation confers subsequent tolerance to organ, tissue, or cellular grafts which are genetically identical or closely matched to the bone marrow previously engrafted.

Beyond transplantation, the ability to establish a successful allogeneic or xenogeneic chimeric hematopoietic system or to reestablish a syngeneic or autologous hematopoietic system can provide cures for various other diseases or disorders which are not currently treated by bone marrow transplantation because of the morbidity and mortality associated with GHVD. Autoimmune diseases involve attack of an organ or tissue by one's own immune system. In this disease, the immune system recognizes the organ or tissue as a foreign. However, when a chimeric immune system is established, the body relearns what is foreign and what is self. Establishing a chimeric immune system as disclosed can simply halt the autoimmune attack causing the condition. Also, autoimmune attack may be halted by reestablishing the victim's immune system after immunosuppression and cytoreduction or after immunodestruction with syngeneic or autologous cell compositions as described hereinbefore. Autoimmune diseases which may be treated by this method include, for example, type I diabetes, systemic lupus erythematosus, multiple sclerosis, rheumatoid arthritis, psoriasis, colitis, and even Alzheimers disease. The use of the FC and HSC can significantly expand the scope of diseases which can be treated using bone marrow transplantation.

Because a chimeric immune system includes hematopoietic cells from the donor immune system, deficiencies in the recipient immune system may be alleviated by a nondeficient donor immune system. Hemoglobinopathies such as sickle cell anemia, spherocytosis or thalassemia and metabolic disorders such as Hunters disease, Hurlers disease, chronic granulomatous disease, ADA deficiency, and enzyme defects, all of which result from deficiencies in the hematopoietic system of the victim, may be cured by establishing a chimeric immune system in the victim using purified donor hematopoietic FC and donor HSC from a normal donor. The chimeric immune system should preferably be at least 10% donor origin (allogeneic or xenogeneic).

The ability to establish successful xenogeneic chimerism can provide methods of treating or preventing pathogen-mediated disease states, including viral diseases in which species-specific resistance plays a role. For example, AIDS is caused by infection of the lymphohematopoietic system by a retrovirus (HIV). The virus infects primarily the CD4⁺ T cells and antigen presenting cells produced by the bone marrow HSC. Some animals, such as, for example, baboons and other nonhuman primates, possess native immunity or resistance to AIDS. By establishing a xenogeneic immune system in a human recipient, with a baboon or other AIDS resistant and/or immune animal as donor, the hematopoietic system of the human recipient can acquire the AIDS resistance and/or immunity of the donor animal. Other pathogen-mediated disease states may be cured or prevented by such a method using animals immune or resistant to the particular pathogen which causes the disease. Some examples include hepatitis A, B, C, and non-A, B, C hepatitis. Since the facilitating cell plays a major role in allowing engraftment of HSC across a species disparity, this approach will rely upon the presence of the facilitating cell in the bone marrow inoculum.

The mobilized or enriched cells of the present invention also provide methods of practicing gene therapy. It has recently been shown that sometimes even autologous cells which have been genetically modified may be rejected by a recipient. Utilizing mobilized cells of the present invention, a chimeric immune system can be established in a recipient using hematopoietic cells which have been genetically modified in the same way as genetic modification of other cells being transplanted therewith. This will render the recipient tolerant of the genetically modified cells, whether they be autologous, syngeneic, allogeneic or xenogeneic.

It will be appreciated that the present invention discloses methods of mobilizing FC and HSC in the peripheral blood of a subject, methods of selectively enriching FC and HSC populations in culture, methods of selectively harvesting such cells, and cellular compositions comprising purified FC and HSC harvested from mobilized blood or enriched cell cultures.

Whereas particular embodiments of the invention have been described hereinbefore, for purposes of illustration, it would be evident to those skilled in the art that numerous variations of the details may be made without departing from the invention as defined in the appended claims. All references cited in the foregoing discussion are hereby incorporated by reference in their entirety.

### 6. EXAMPLE 1

### FL and G-CSF Mobilized FC and HSC into Donor Peripheral Blood which Repopulated a Recipient with Aplasia

6.1. Materials and Methods

6.1.1. Animals

Four to six week old B10.BR SgSnJ (H-2K^{k}) and C57BL/10SnJ (H-2K^{b}) mice were purchased from Jackson Laboratory, Bar Harbor, Me. The animals were housed in a pathogen-free animal facility at the Institute for Cellular Therapeutics, Allegheny University of the Health Sciences, Philadelphia, Pa., and cared for according to specific Allegheny University and National Institutes of Health animal care guidelines.

6.1.2. Reagents

FL and G-CSF were obtained from Immunex Corp. (Seattle, Wash.) and Amgen, Inc. (Thousand Oaks, Calif.), respectively. These agents were diluted to the appropriate concentrations with 0.9% saline prior to in vivo administration.

6.1.3. Administration of FL and G-CSF

Bb 10.BR mice were divided into four groups (n≧6 per experimental group, n=4 for control group). The mice were injected subcutaneously with FL at a daily dose of 10 μg from day 1 to 10 (group A), G-CSF at a daily dose of 7.5 μg from day 4 to 10 (group B), or a combination of FL and G-CSF with the doses and duration of treatment as indicated above (group C). Control animals received saline (group D). FL and G-CSF were diluted daily with 0.9% saline to a final injection volume of 0.5 ml per animal. Animals were injected subcutaneously in the morning of each day.

6.1.4. Flow Cytometry

Peripheral blood was obtained daily from day 1 to day 11 from two animals of each group. White blood cells were counted using the hemocytometer. Whole blood was stained using the following monoclonal antibodies (mAb): CD8-FITC, B220-FITC, Mac1-FITC, GR1-FITC, αβTCR-FITC, γδTCR-FITC, CD8-PE, SCA1-PE, and C-kit-Bio (all purchased from Pharmingen, San Diego, Calif.). After incubation with mAb for 30 minutes at 4° C., cells were washed twice and counterstained with streptavidin-APC (Becton-Dickinson, San Jose, Calif.) for 15 minutes. Red blood cells were lysed using Facs lysing solution (Becton-Dickinson) and samples were analyzed within 34 hours by flow cytometry using a FACSCalibur (Becton-Dickinson). FC were defined as cells positive for CD8 but negative for αβTCR as well as γδTCR-FITC (FIGS. 1A and 1B) and hematopoietic stem cells (HSC) as C-kit⁺ and SCA⁺ but lineage⁻. The calculation of absolute numbers of FC and HSC were performed based on the percentage of these populations on total events and the whi te blood cell count.

6.1.5. Repopulation of Recipients

In order to assess the engraftment potential of mobilized PB, allogeneic C57B1/10SNJ mice were lethally irradiated with a single dose of 950cGy total body irradiation using a Cesium source (Nordion, Ontario, Canada) and transplanted via the lateral tail vein with mobilized blood containing 1×10⁶ or 5×10⁶ white blood cells from donors previously treated with FL alone, G-CSF alone, or saline, or 1×10⁶, 2×10⁶, or 5×10⁶ white blood cells from donors previously treated with FL plus G-CSF. Reconstituted animals were monitored daily for incidence of failure of engraftment, and as an indication of the ability of mobilized blood to engraft and repopulate recipients with irradiation-induced aplasia. Six weeks after transplantation, peripheral blood was obtained from recipients and stained with Mab specific for donor (H-2^{b}) and recipient (H-2K^{k}) mHc class I to assess engraftment of HSC and the level of donor chimerism.

6.2. Results

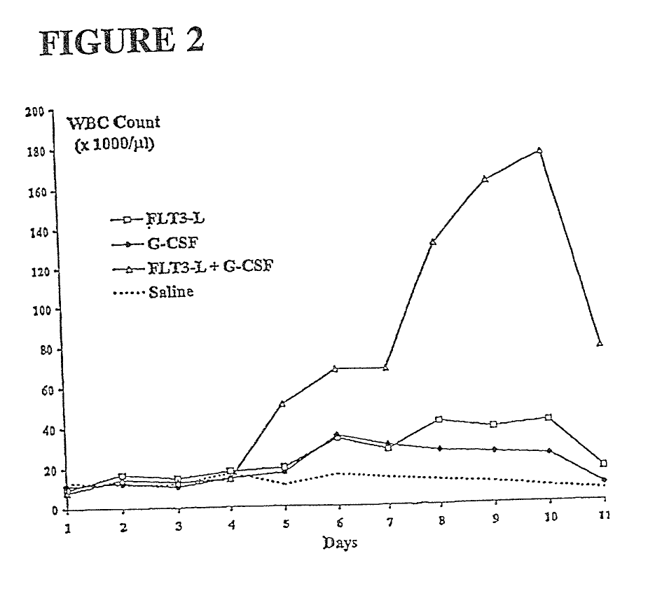

Animals were treated with FL, G-CSF, FL plus G-CSF or saline and their PB analyzed by flow cytometry for the presence of FC (CD8⁺/αβTCR⁻/γδTCR⁻) and HSC (c-kit⁺/SCA⁺/lineage⁻). The number of white blood cells in peripheral blood was most effectively increased by the combin ation of G-CSF and FL (FIG. 2). Treatment with FL plus G-CSF was also most effective in mobilizing both FC and HSC, resulting in a 24-fold increase in the absolute number of FC in PB, and a 287-fold increase in the absolute number of HSC. Peak levels were reached on day 10. Treatment with FL alone resulted in a 6-fold increase in FC, with a peak at day 6. In contrast, treatment with G-CSF alone resulted in only a 3-fold increase of FC. The time course for FC mobilization was similar to that of HSC (FIGS. 3A and 3B). The absolute number of T cells did not increase significantly, indicating that the mobilization was specific for FC and HSC.

Mobilized FC and HSC exhibited repopulating potential, since MHC-disparate mice were routinely rescued from irradiation-induced aplasia by mobilized peripheral blood containing 5×10⁶ white blood cells from donor group A, B, and C, while recipients reconstituted with peripheral blood from untreated donors died within 2 weeks (Table 1).

TABLE 1

| | | White | | | Mean Donor |
|---|---|---|---|---|---|
| Donor | Donor | Blood Cell | Number of | Survival | Chimerism |
| Group | Treatment | Dose | Recipients | (Days) | (%) |
| A | FL | 1 × 10⁶ | 3 | 17; 29; | 90 |
| | | | | >70 | |
| | | 5 × 10⁶ | 3 | 10; >70 | 87 |
| | | | | (2×) | |
| B | G-CSF | 1 × 10⁶ | 3 | 36; 49; | 91 |
| | | | | >70 | |
| | | 5 × 10⁶ | 3 | >70 (3×) | 94 |
| C | FL + G-CSF | 1 × 10⁶ | 4 | 17; 31; | 94 |
| | | | | 41; >70 | |
| | | 2 × 10⁶ | 3 | >70 (3×) | 93 |
| | | 5 × 10⁶ | 3 | >70 (3×) | 93 |
| D | Saline | 1 × 10⁶ | 2 | 10; 11 | — |
| | | 5 × 10⁶ | 2 | 12; 14 | — |

In conclusion, FC and HSC were mobilized into the PB in substantial numbers by a combination of FL and G-CSF with a peak on day 10. Therefore, collection of PB at the appropriate time following mobilization includes the maximum number of both FC and HSC. In striking contrast, G-CSF or FL alone was much less effective. The superior survival of allogeneic animals transplanted with mobilized PB demonstrates the repopulating potential of both FC and HSC.

### 7. EXAMPLE 2

### Effect of FL and G-CSF on Expansion and Mobilization of FC and HSC in Mice: Kinetics and Repopulating Potential

In the present study we evaluated the ability of FL alone, G-CSF alone or the two in combination to mobilize cells of FC phenotype in the periphery and to study the kinetics of FC and HSC mobilization to define optimal timing for the collection of both populations. Both growth factors showed a highly significant synergy on the mobilization of FC and HSC. The kinetics for mobilization were similar for FC and HSC, with a peak occurring on day 10. G-CSF alone was not efficient at mobilizing FC. We further analyzed the distribution of FC and HSC in hematopoietic sites such as spleen and bone marrow of growth factor-treated mice at different time points. A dramatic expansion of both FC and HSC was observed in spleen of FL and FL+G-CSF-treated animals while no significant changes were detectable in spleen of mice injected with G-CSF alone. In bone marrow of animals treated with FL alone the frequency of FC showed a 5-fold increase. This phenomenon was not observed in animals that received G-CSF alone or in combination with FL. The engraftment-potential of HSC and FC mobilized by FL and FL+G-CSF into fully ablated MHC-disparate recipients was superior to that for donors treated with G-CSF alone.

7.1. Materials and Methods

7.1.1. Animals

Four- to 6-week-old male C57BL/10SnJ (B10, H-2^{b}) and B10.BR.SgSnJ (B10.BR,H-2^{k}) mice were purchased from the Jackson Laboratory (Bar Harbor, Me.). Animals were housed in a barrier animal facility at the Institute for Cellular Therapeutics, Allegheny University of the Health Sciences, Philadelphia, Pa. and cared for according to specific Allegheny University and National Institutes of Health animal care guidelines.

7.1.2. Growth Factors

Recombinant human FL was obtained from Immunex (Immunex Corp., Seattle, Wash.) and diluted in saline (Sigma, St. Louis, Mo.) supplemented with 0.1% mouse serum albumin (MSA; Sigma, St. Louis, Mo.) at a concentration of 100 μg/ml. Recombinant human G-CSF was obtained from Amgen (Amgen Inc., Thousand Oaks, Calif.). Growth factors were diluted in saline prior to injections to a total volume of 500 μl and B10.BR mice were injected once daily subcutaneously (SC). Mice received either 10 μg FL/day alone from day 1 to 10, 7.5 μg CSF/day alone from day 4 to 10 or a combination of FL and G-CSF at the aforestated dosages and durations. Brasel, K. et al., Blood 88:2004, 1996; Molineux, G. et al., Blood 89:3998, 1997; Brasel, K. et al., Blood 90:3781, 1997. Control animals were injected with saline only.

7.1.3. Tissues

PB was obtained daily from the tail vein of growth factor-treated animals. After individual counts of peripheral blood mononuclear cells (PBMC) with a hemocytometer, cells were stained for flow cytometric analysis to study the kinetics of FC and HSC mobilization. In separate experiments peripheral blood (PB) was collected on days 0, 7 and 10 from growth factor-treated anesthetized animals via cardiac puncture into heparinized tubes and pooled for each group for reconstitution of allogeneic recipients. At the same time points, spleens and long bones were harvested and single cell suspensions were prepared for flow cytometric analysis. Splenocytes were isolated by gently flushing the organ with media 199 (MEM; Life Technologies, Rockville, Md.). Red blood cells (RBC) were lysed using ammonium chloride lysing buffer (ACK; prepared in our laboratory). Bone marrow was harvested from tibiae and femurs as described previously (Ildstad, S. T. and Sachs, D. U., Nature 307:168, 1984). Briefly, bones were flushed with MEM. Bone marrow cells were resuspended and filtered through a sterile nylon mesh. After centrifugation, cells were resuspended in MEM and counted.

7.1.4. Monoclonal Antibodies

Anti-U-2Kb-PE (AF6-88.5); anti-H-2K^{k}-FITC and -Biotin (36-7-5); anti-GR1-FITC (RB6-8C5); anti-Mac-1 (CD11b)-FITC (M1/70); anti-CD8a-FITC and -APC (53-6.7); anti-CD11b-FITC (M1/70); anti-B220/CD45R-FITC (RA3-6B2); anti-αβTCR-FITC and -PE (H57-597), anti-γδTCR-FITC and -PE (GL3); anti-NK1.1-PE (PK 136); anti-Sca-1 (Ly6A/E)-PE (D7) and anti-c-kit (CD117)-Biotin (2B8) were purchased from Pharmingen (Pharmingen, San Diego, Calif.). Streptavidin-APC was purchased from Becton Dickinson (Becton Dickinson, Mountain View, Calif.).

7.1.5. Detection of FC and HSC by Flow Cytometry

The mobilization kinetics of FC and HSC in PB were analyzed daily for individual animals. Aliquots of 100 μl PB were incubated with mAbs for 30 minutes on ice. Cells were washed twice in FACS medium (prepared in laboratory). Cells labeled with biotinylated mAb were counterstained with streptavidin-APC for 15 minutes. Red blood cells were lysed using ammonium chloride lysing buffer. PBMC were washed twice and fixed in 2% paraformaldehyde (Tousimis Research Corporation, Rockville, Md.). Flow cytometric analysis was performed using a FACSCalibur (Becton Dickinson) as described previously. Kaufman, C. L. et al., Blood 84:2436, 1994. For analysis of FC and HSC, a minimum of 1×10⁵ events were collected. FC were defined as cells residing in a wide lymphoid gate with a dim to intermediately positive expression of CD8 but negative for expression of αβTCR and γδTCR. For enumeration of HSC, cells positive for Sca-1 (Ly -6A/E) and negative for lineage markers (lin⁻) were gated. Gated cells were then analyzed for their expression of c-kit (CD 117). Lin⁻/Sca-1⁺/c-kit⁺ cells were defined as HSC. Statistical analysis of flow data was performed using CELL Quest Software, Version 3.0.1 (Becton Dickinson). The percentage of FC and HSC of total PBMC was determined and the absolute number of FC and HSC per Al blood was calculated based on individual PBMC counts. In addition, the percentage of FC and HSC in spleen and bone marrow was determined at different time points under treatment with FL and/or G-CSF.

7.1.6. Reconstitution of Allogeneic Recipients with Mobilized PB

To investigate the repopulating potential of FC and HSC in mobilized PB, allogeneic B10 mice were lethally irradiated with a single dose of 950 cGy total body irradiation (TBI) (117.18 Cgy/min) from a cesium source (Nordion, Ontario, Canada). On day 7 or 10 of mobilization, PB was obtained from B10.BR mice, pooled for each treatment group and counted. Three to 5 hours following irradiation, animals were reconstituted with mobilized whole blood containing 1×10⁶, 2.5×10⁶ or 5×10⁶ PBMC diluted in MEM to a total volume of 1 ml via the lateral tail vein. Radiation controls as well as control animals reconstituted with equal numbers of PBMC from unmobilized PB were prepared. Reconstituted animals were monitored daily to detect failure of engraftment as indicated by excessive body weight loss and survival was calculated based on the life-table method. In addition, PBMC counts were performed 10, 20 and 30 days following reconstitution.

7.1.7. Characterization of Chimeras by Flow Cytometry

Thirty days and 6 months after reconstitution, recipients were analyzed for evidence of donor cell engraftment by flow cytometry to detect the percentage of PBMC bearing H-2K^{b} (recipient) and H-2K^{k} (donor) markers. PB was collected from the tail vein into heparinized vials. After thoroughly mixing, 100 μl PB was incubated with anti-H-2K^{b}-PB and anti-H-2K^{k}-FITC mAb for 30 minutes on ice. RBC were lysed using ammonium chloride lysing buffer. PBMC were washed twice and fixed in 2% paraformaldehyde (Tousimis Research Corporation). Lymphocytes, granulocytes and monocytes were gated based on forward and side scatter and analyzed for H-2Kb or H-2K^{k} expression. PB from unmanipulated B10 and B10.BR mice served as controls.

To confirm HSC engraftment, the presence of multilineage chimerism was assessed using three-color flow cytometry 6 months after reconstitution. PB was obtained and stained with FITC- and PE-labeled lineage mAbs and biotinylated anti-2K^{k} mAb, counterstained with streptavidin-APC, as described above.

7.1.8. Statistical Analysis

Statistical analyses were performed using unpaired two-tailed Student's t-test and p-values <0.05 were considered as significant. The 6-month survival of transplanted animals was assessed using Kaplan-Meier estimates and survival of different groups was compared using Wilcoxon test.

7.2. Results

7.2.1. Kinetics of Mobilization of FC and HSC in PB

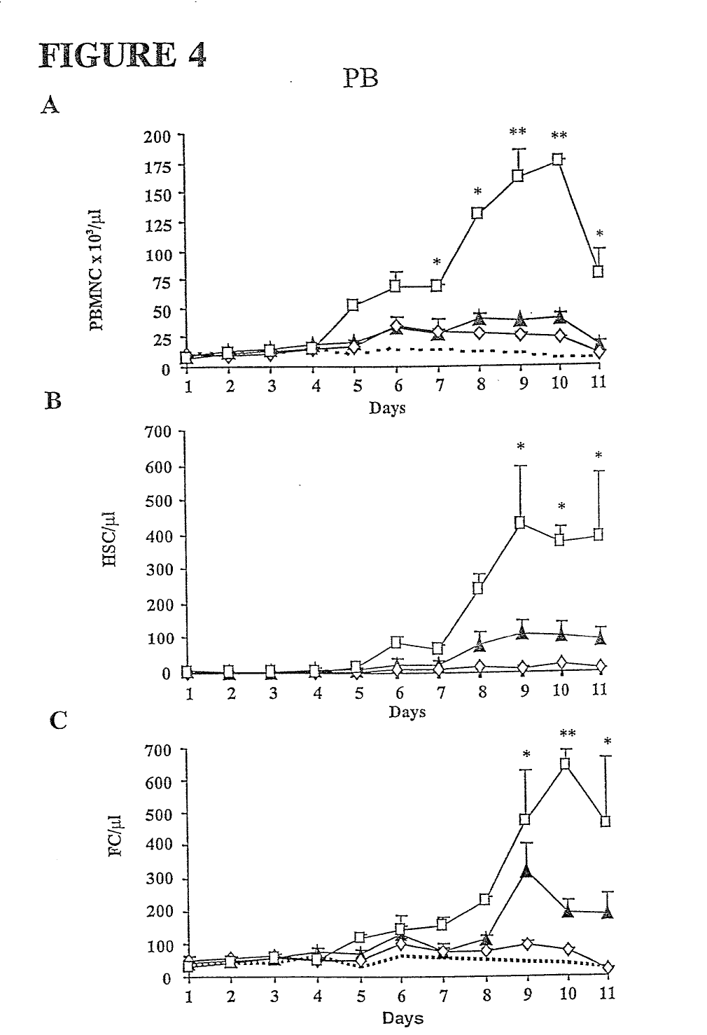

We evaluated the effect on the total number of PBMC in PB after administration of FL alone (day 1 to 10), G-CSF alone (day 4 to 10) or a combination of both growth factors. Animals treated with G-CSF and FL alone showed a 3-fold and 4-fold increase of PBMC, respectively (FIG. 4A). Combined administration of both growth factors showed a synergistic effect, since PBMC increased significantly and a maximum (22-fold increase) was observed on day 10. PBMC of animals injected with carrier only remained at baseline levels.

To assess the potential of growth factor-administration on mobilization of FC and HSC, the absolute number of FC and HSC under treatment with FL alone, G-CSF alone and a combination of FL and G-CSF was determined (FIG. 4B and 4C). While G-CSF alone resulted in a 17-fold increase of primitive HSC, only a modest effect on the mobilization of FC was noted. In contrast, FL as a single agent caused a 7-fold increase of FC and 36-fold increase of HSC, respectively, and peak levels for both populations occurred on day 9. A maximal elevation of both FC and primitive HSC was detectable when both growth factors were combined. An increase of HSC was detectable on day 6 and a plateau reflecting a more than 200-fold increase or an absolute number of approximately 400 HSC/μl PB was reached from day 9 to 11. The number of cells of FC phenotype increased on day 5 and a peak level representing a 21-fold increase was observed on day 10. In contrast to HSC, the number of FC declined rapidly after day 10.

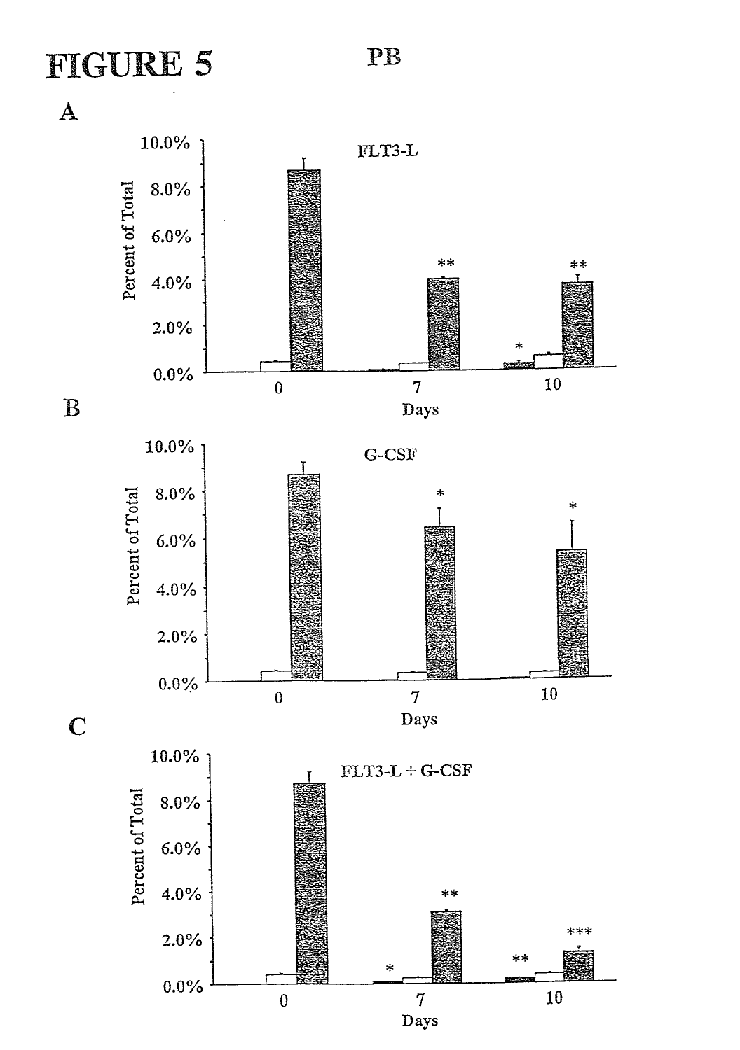

Since the majority of cells in PB after FL+G-CSF treatment were neutrophilic granulocytes, the relative percentage of CD8⁺ T cells decreased significantly from 8.7% on day 0 to 1.3% on day 10 (FIGS. 5 A-C). In striking contrast, the percentage of FC remained at a constant level, while an increase in the percentage of HSC from 0.01% on day 0 to 0.36% on day 10 was observed. A similar observation was made when mice were treated with FL alone. CD8⁺ T cells in G-CSF treated animals showed only a slight decrease (8.7% to 5.4%) and no significant changes in the percentage of FC and HSC were observed.

7.2.2. Distribution of FC and HSC in Spleen and Bone Marrow of Mice Treated with Growth Factors

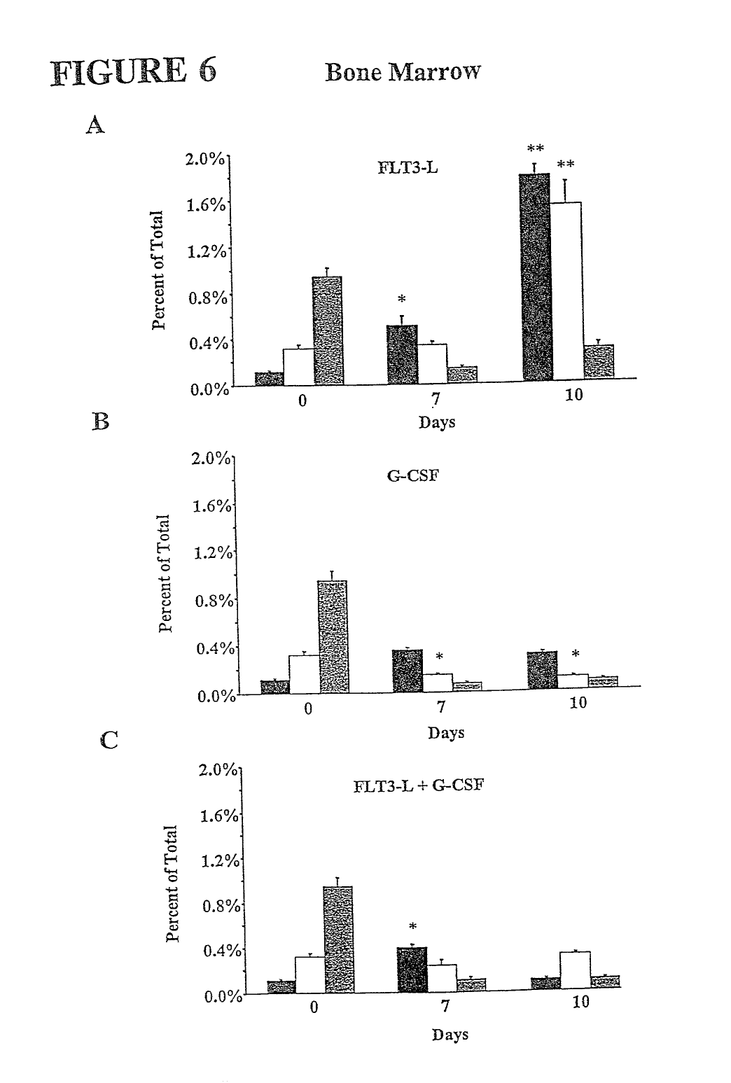

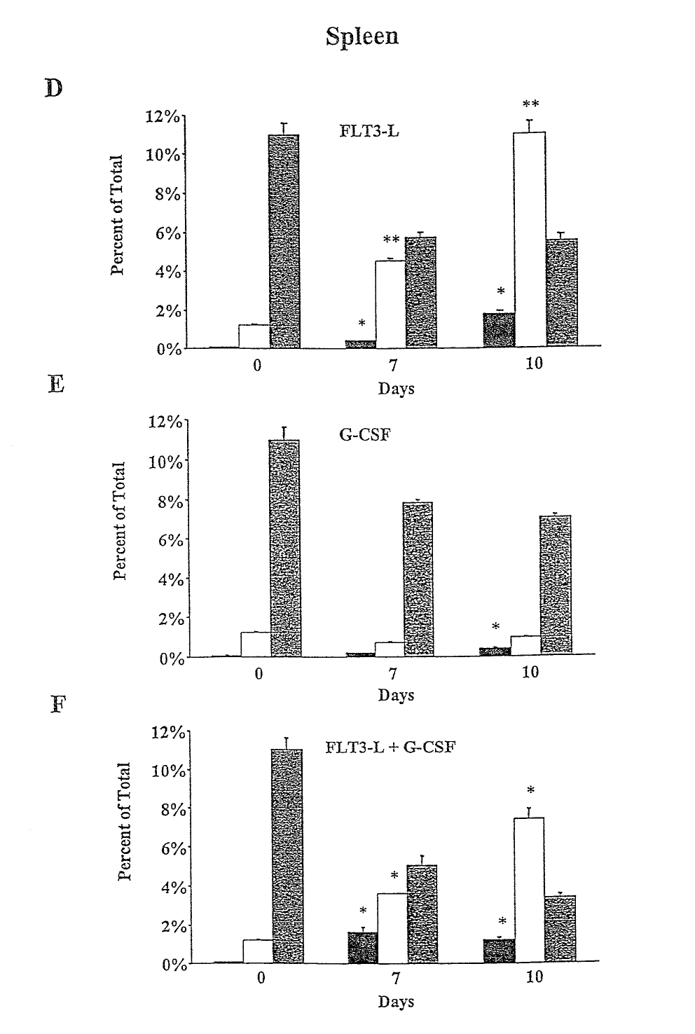

To address whether the observed increase in absolute numbers of FC and HSC in PB was due to mobilization of preexisting cells or due to de novo hematopoiesis, splenocytes and bone marrow cells from FL, G-CSF and FL+G-CSF treated mice were analyzed by flow cytometry and percentages of FC and HSC were determined. Mobilization of mature cells from the bone marrow into the periphery occurred in all growth factor treated animals as indicated by a significant reduction of the percentage of CD8⁺ T cells (FIGS. 6A, B and C). In the bone marrow of animals that received G-CSF alone only a marginal increase in the percentage of HSC was present, while the frequency of FC decreased significantly during mobilization. In animals treated with FL and G-CSF a significant increase in the percentage of HSC was observed on day 7. However, on day 10 of mobilization the frequency of HSC in bone marrow decreased to baseline levels. Interestingly,-in mice treated with FL alone an 18-fold and 5-fold increase in the percentage of HSC and FC was detected, respectively, indicating proliferation and/or lack of mobilization of FC and HSC in the absence of G-CSF.

In spleen, the frequency of both FC and HSC increased over time under treatment with FL alone or FL in combination with G-CSF (FIGS. 6D and F). Cells of FC phenotype increased significantly from 1% on day 0 to 11% on day 10 in animals treated with FL alone and from 1% on day 0 to 7% on day 10 in animals that received a combination of both growth factors. The percentage of primitive HSC in spleen of FL and FL+G-CSF treated mice showed a 20-fold (0.09% on day 0 to 1.79% on day 10) and an 18-fold increase (0.09% on day 0 to 1.62% on day 7), respectively. In striking contrast, under treatment with G-CSF alone the percentage of FC remained unchanged over time, while the frequency of HSC was slightly elevated (FIG. 6E).

7.2.3. Short Term Engraftment Potential of Mobilized PBMC in Allogeneic Recipients

To determine the short term engraftment-potential of HSC and FC mobilized by treatment with FL, G-CSF or FL+G-CSF, allogeneic B10 mice were lethally irradiated and reconstituted with whole PB containing varying numbers of PBMC. Recipients were transplanted with either 1×10⁶, 2.5×10⁶ or 5×10⁶ PBMC from donors treated with growth factors for 7 or 10 days. The cell number of mobilized HSC and FC per kg body weight of recipients is shown in Table 2.

TABLE 2
HSC and FC dose per kg body weight injected into lethally irradiated (950 cGy TBI)
C57BL/10SnJ mice.

| Donor | PBMC | HSC Dose/kg | FC Dose/kg | HSC Dose/kg | FC Dose/kg |
|---|---|---|---|---|---|
| Treatment | Dose/Recipient | [×105] | [×105] | [×105] | [×105] |
| FL | 1 × 106 | 0.06 ± 0.00 | 0.96 ± 0.07 | 0.63 ± 0.02 * | 2.10 ± 0.07 * |
| | 2.5 × 106 | 0.17 ± 0.00 | 2.50 ± 0.00 | 1.59 ± 0.24 * | 5.28 ± 0.79 |
| | 5 × 106 | 0.34 ± 0.02 | 5.12 ± 0.26 | 2.84 ± 0.25 * | 9.41 ± 0.83 * |
| G-CSF | 1 × 106 | 0.06 ± 0.00 | 1.06 ± 0.07 | 0.18 ± 0.01 | 0.93 ± 0.07 |
| | 2.5 × 106 | 0.17 ± 0.01 | 2.75 ± 0.09 | 0.43 ± 0.02 | 2.22 ± 0.09 |
| | 5 × 106 | 0.33 ± 0.04 | 5.43 ± 0.64 | 0.92 ± 0.24 | 4.77 ± 1.23 |
| FL + G-CSF | 1 × 106 | 0.29 ± 0.01 | 0.88 ± 0.02 | 0.64 ± 0.03 * | 1.21 ± 0.06 * |
| | 2.5 × 106 | 0.65 ± 0.04 * | 1.95 ± 0.13 * | 1.71 ± 0.07 * | 3.24 ± 0.13 * |
| | 5 × 106 | 1.27 ± 0.06 * | 3.80 ± 0.19 * | 3.38 ± 0.18 * | 6.42 ± 0.34 * |
| B10.BR donors were treated with either FL alone, G-CSF alone or FL plus G-CSF. PB was collected on day 7 or day 10, pooled for each group and recipients were reconstituted with either 1 × 10⁶, 2.5 × 10⁶, or 5 × 10⁶ PBMC. The numbers of HSC (lineage ⁻/Sca-1⁺/c-kit⁺) and FC (CD8⁺/αβTCR⁻/γδTCR⁻) per kg body weight of recipients was calculated based on flow | | | | | |
| # cytometric analysis and is expressed as the mean ± SD. The cell numbers at which the 30-day survival reached > 80% are marked (*). | | | | | |

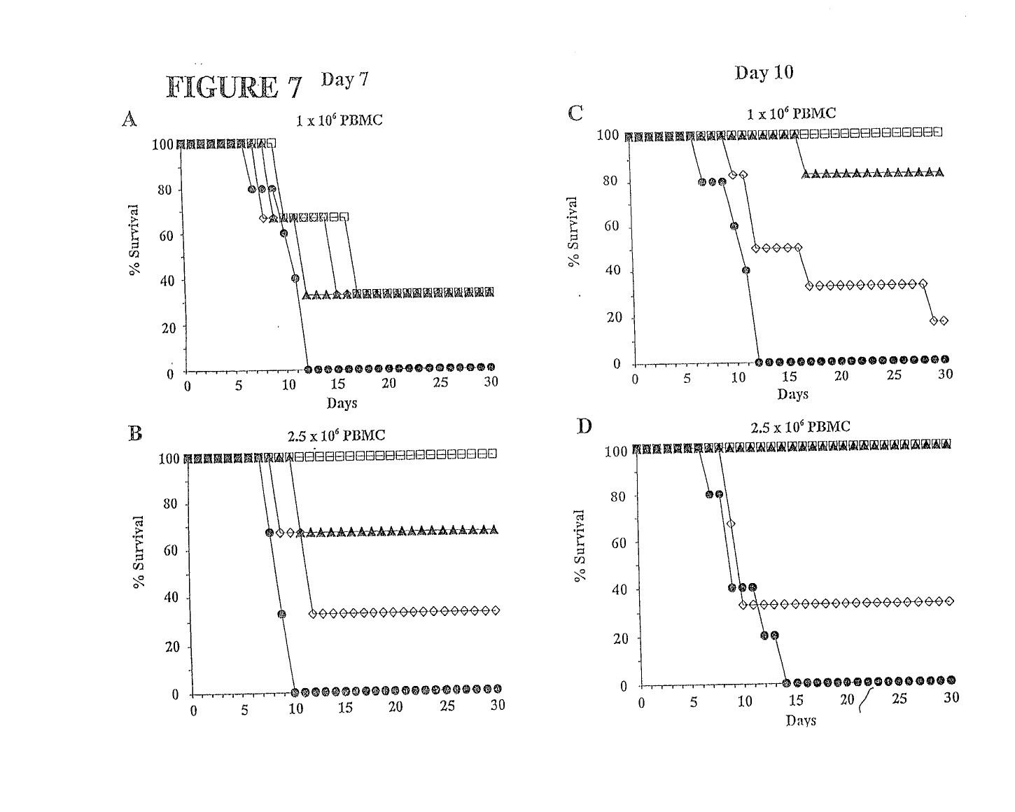

Control animals received equal amounts of PBMC from untreated B10.BR mice. The 30-day survival of transplanted animals as a function of PBMC dose and time-point of collection of PB is shown in FIGS. 7A-7D. Animals reconstituted with 1×10⁶ PBMC collected on day 7 from donors treated with FL, G-CSF or FL+G-CSF showed a 33% survival at day 30. Control animals injected with 1×10⁶ PBMC from untreated donors died within 12 days from irradiation-induced aplasia (FIG. 7A). At a cell dose of 2.5×10⁶ PBMC, the 30-day survival rate increased to 67% after treatment with FL alone and 100% after treatment with FL+G-CSF. No significant difference between both cell doses was observed for the G-CSF treatment group and control group (FIG. 7B). The 30-day survival of irradiated recipients was superior when PB from FL or FL+G-CSF treated animals was collected after 10 days of growth factor-administration. As few as 1×10⁵ PBMC mobilized with FL alone rescued from irradiation-induced aplasia more than 80% of recipients, while with FL+G-CSF 100% of transplanted animals survived (FIG. 7C and Table 3). At a PBMC dose of 2.5×10⁶ 100% of animals transplanted with FL and FL+G-CSF mobilized PB were alive after 30 days (FIG. 7D). When 1×10⁶ and 2.5×10⁶ PBMC mobilized with G-CSF as a single agent were given, the 30-day survival rate was only 20% and 33%, respectively. All control animals injected with PB from untreated donors died within 14 days from TBI-induced aplasia. Irradiation controls that received 950 cCy TBI without PBMC injection died within 10 days (data not shown). When the dose of PBMC collected on day 7 or day 10 was further increased to 5×10⁶ no further improvement of the 30-day survival rate was observed for all treatment groups (data not shown).

TABLE 3

| | Donor | | Survival |
|---|---|---|---|
| Donor Group | Treatment | PBMC Dose | Rate (%) |
| A | FL | 1 × 10⁶ | 83 |
| | | 5 × 10⁶ | 100 |
| B | G-CSF | 1 × 10⁶ | 17 |
| | | 5 × 10⁶ | 33 |
| C | FL + G-CSF | 1 × 10⁶ | 100 |
| | | 5 × 10⁶ | 100 |
| D | Saline | 1 × 10⁶ | 0 |
| | | 5 × 10⁶ | 0 |

To study the time course of HSC and FC engraftment, PBMC from individual animals transplanted with mobilized PB collected on day 10 were counted 10, 20 and 30 days following reconstitution. Engraftment, def ined as a PBMC count of ≧500 PBMC/μl was observed in ≧67% of recipients 20 days after reconstitution with 1×10⁶ and 2.5×10⁶ PMBC from FL and FL+G-CSF treated donors (Table 4). After 30 days 100% of these animals had a PBMC count of ≧500 PBMC/μl. When 5×10⁶ PBMC from FL and FL+G-CSF treated animals were injected engraftment occurred as early as on day 10 in ⅓ and 3/3 recipients, respectively. In striking contrast, animals reconstituted with equal amounts of PBMC from G-CSF treated or unmanipulated donors did not present PBMC counts of ≧500 PBMC/μl at any of the time points tested (data not shown).

TABLE 4
Time to engraftment of mobilized PBMC from B10.BR mice into lethally irradiated C57BL/10SnJ mice.

| | | Time to Engraftment: Number of | | | | | |
|---|---|---|---|---|---|---|---|
| PBMC | Donor | Animals with ≧ 500 PBMC/μl | | | Median PBMC Count (Range) [PBMC/μl] | | |
| Dose | Treatment | Day 10 | Day 20 | Day 30 | Day 10 | Day 20 | Day 30 |
| 1 × 10⁶ | FL | 0/3 | 2/3 | 3/3 | <100 | 560 (200-1,300) | 1,520 (750-4,800) |
| | FL + G-CSF | 0/3 | 3/3 | 3/3 | <100 | 850 (620-1,080) | 750 (550-1,350) |
| 2.5 × 10⁶ | FL | 0/3 | 3/3 | 3/3 | <100 | 920 (520-4,480) | 3,500 (1,100-5,000) |
| | FL + G-CSF | 0/3 | 2/3 | 3/3 | <100 | 740 (420-1,460) | 1,900 (1,500-5,600) |
| 5 × 10⁶ | FL | 1/3 | 2/3 | 3/3 | 220 (180-540) | 620 (460-1,480) | 4,000 (2,300-4,960) |
| | FL + G-CSF | 3/3 | 3/3 | 3/3 | 900 (800-920) | 1,360 (1,240-3,960) | 5,800 (5,200-7,100) |

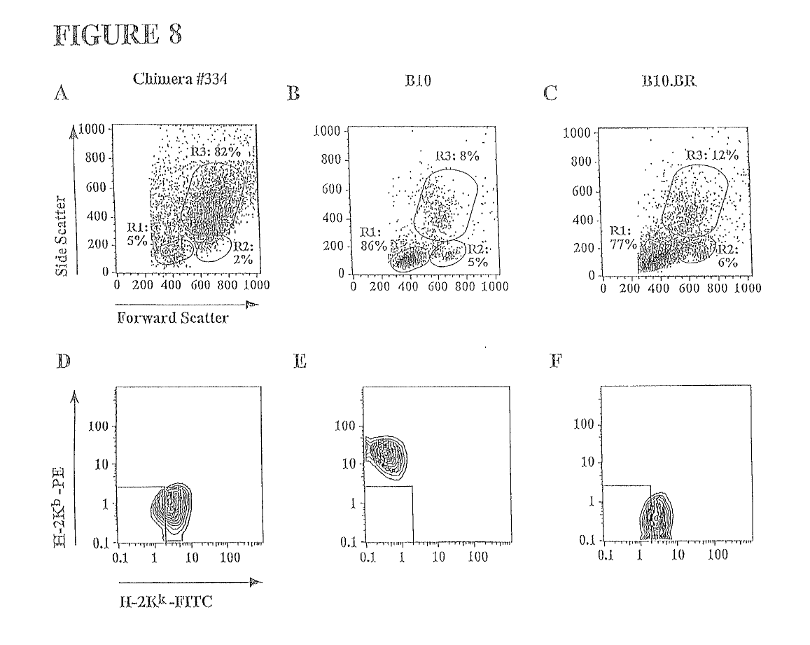

Flow cytometric analysis of PB obtained from transplanted animals 30 days following reconstitution was performed and the lineage derivation of PBMC was determined based on cell size and granularity. To distinguish the PBMC of donor origin from the radio-resistant or repopulating cells of host origin, PB was stained with nAbs specific for host (H-2K^{b}) and donor (H-2K^{k}) MHC class I antigen. In engrafted recipients 91.2%±4.0% of PBMC were located in the granulocyte gate, while 5.6%±3.1% and 0.5%±0.1% of PBMC resided in the lymphocyte or monocyte gate, respectively (FIGS. 8 A-F). More than 95% of PBMC were of donor origin.

7.2.4. Long Term Engraftment of Mobilized HSC in Allogeneic Recipients

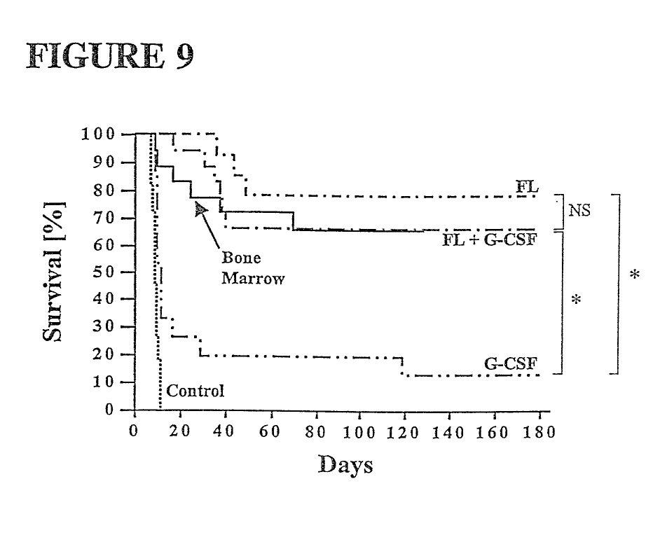

Long-term survival (>6 months) was 79% and 67% in animals transplanted with PBMC from FL and FL+G-CSF treated donors, respectively (FIG. 9). This survival rate was comparable to that of recipients (n=25) reconstituted with 1×10⁶ untreated bone marrow cells from naive B10.BR donors. he majority of recipients reconstituted with mobilized PB developed clinical signs of acute GVHD within 30 to 60 days after transplantation as indicated by diarrhea and loss of body weight. However, GVHD was self-limiting in most of these animals. In striking contrast, long-term survival of animals transplanted with PBMC mobilized with G-CSF alone was significantly lower and the estimated survival after 6 months was only 13%. None of the recipients transplanted with PBMC from carrier-treated B10.BR donors survived for more than 14 days.

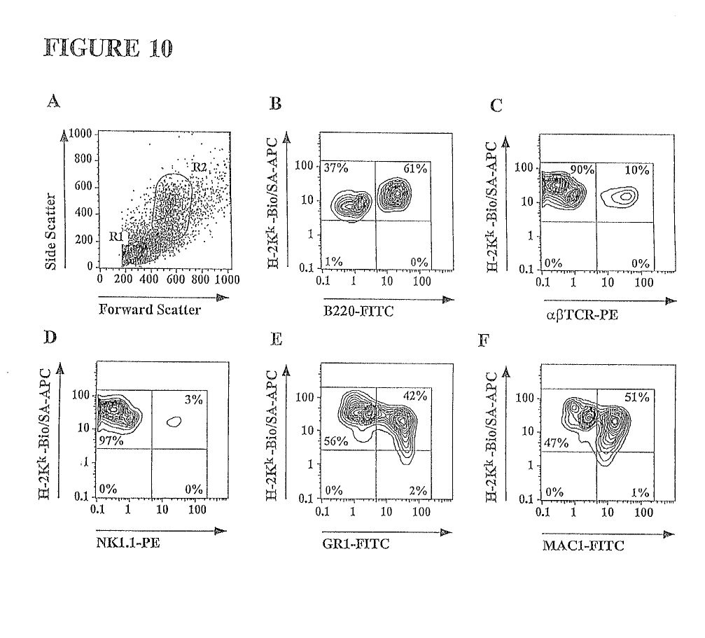

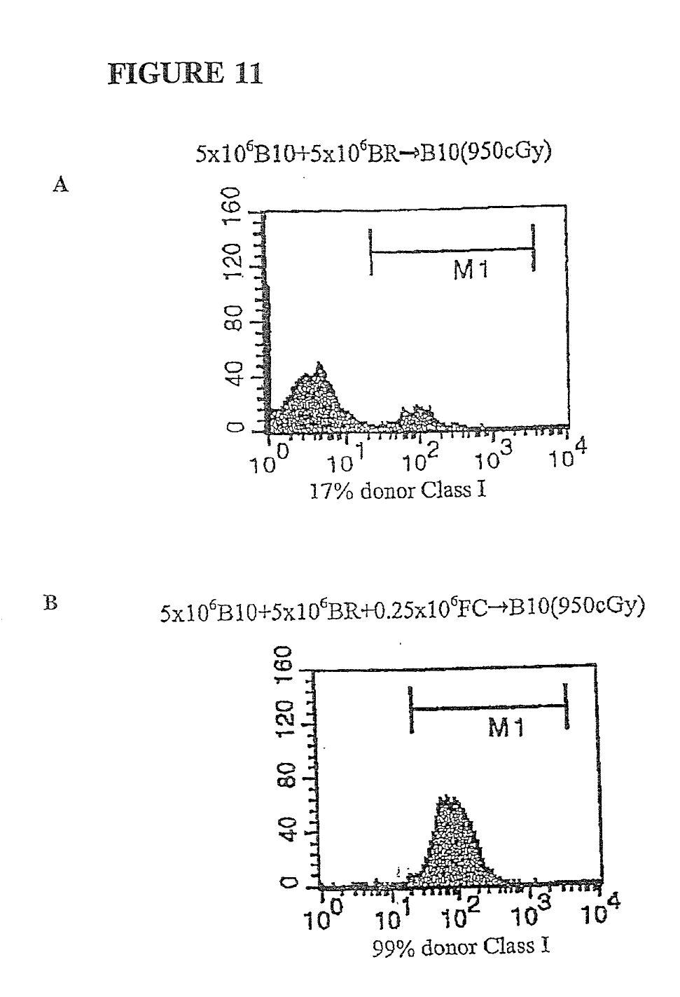

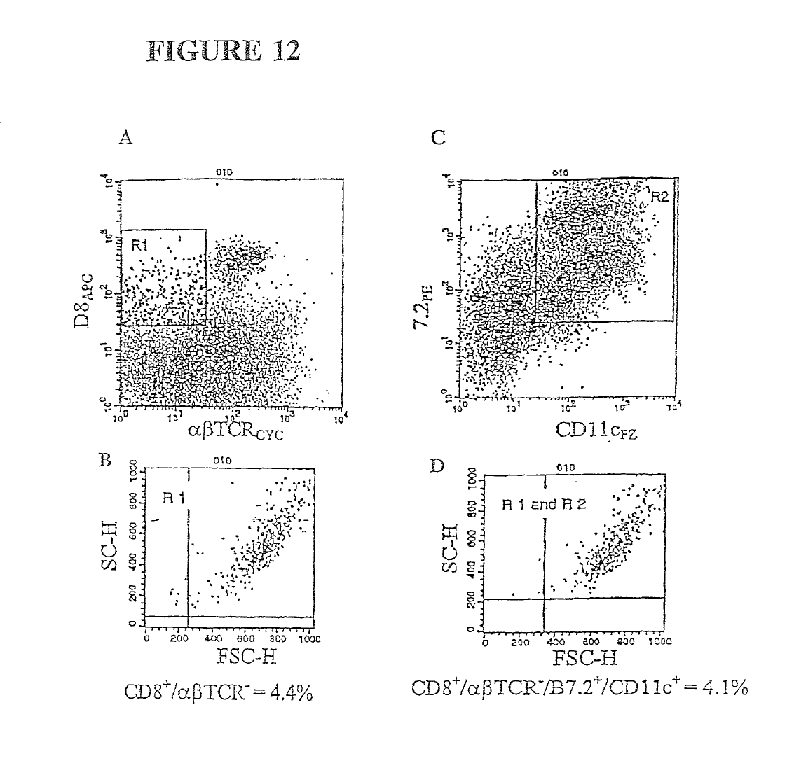

Representative animals transplanted with PB from donors treated with growth factors for 10 days were analyzed after 6 months and the level of donor chimerism as well as the presence of multiple hematopoietic lineages was assessed by flow cytometry using lineage-specific mabs. All long-term surviving animals tested showed >99% donor chimerism and multiple lineages including B-cells, αβTCR⁺ T cells, γδTCR⁺T cells, NK cells, macrophages and granulocytes of donor origin were present in these animals (Table 5 and FIG. 10). Percentages of analyzed donor-derived cell lines was comparable to that of naive B10.BR mice.

TABLE 5
Characterization of long term surviving animals (C57BL/10SnJ) reconstituted with mobilized PB from B10.BR
donors treated with FL alone, G-CSF alone or FL plus G-CSF.

| Donor | | Donor | B-cells | αβTCR⁺ | γδTCR⁺ | NK cells | Granulocytes | Macrophages |
|---|---|---|---|---|---|---|---|---|
| Treatment | N | Chimerism* | (%)⁺ | T-Cells (%) ⁺ | T-cells (%) ⁺ | (%) ⁺ | (%) ^{•} | (%) ^{•} |
| FL | 4 | >99% | 57.5 ± 14.6 | 22.1 ± 8.0 | 0.6 ± 0.1 | 2.3 ± 0.7 | 40.4 ± 5.4 | 35.2 ± 7.5 |
| G-CSF | 4 | >99% | 71.3 ± 0.8 | 15.0 ± 7.4 | 0.5 ± 0.1 | 2.0 ± 0.8 | 41.3 ± 10.5 | 61.9 ± 0.8 |
| FL + G-CSF | 6 | >99% | 48.7 ± 5.4 | 24.5 ± 7.8 | 0.7 ± 0.2 | 2.3 ± 0.4 | 24.4 ± 4.8 | 33.4 ± 8.4 |

PB was obtained from representative recipients 6 months after reconsititution. PBMC stained with lineage- and donor-specific mAbs for three-color flow cytometric analysis.
(*) Level of donor chimerism was calculated on gated lymphocytes with positive staining for donor (H-2K^{k}) but negative staining for recipient MHC class I (H-2K^{b}).
The mean percentage (± SD) of PBMC with positive staining for lineage- and donor-specific mAbs on gated lymphocytes (^{•}) or granulocytes (*) is shown.

7.3. Discussion

It has been previously shown that FL mobilizes large numbers of PBMC into the circulation of mice. Ashihara, E. et al., Exp. Hematol., 23:801a, 1995 (abstract); Brasel, K. et al., Blood, 88:2004, 1996. A maximum effect was observed when FL was injected at a daily dose of 10⁴/μg subcutaneously for 10 days resulting in an increase of PBMC to 4×10⁴/μl. In subsequent experiments a synergistic effect was observed when FL was used in combination with G-CSF. Brasel, K. et al., Blood 90:3781, 1997; Sudo, Y. et al., Blood 89:3 186, 1997. In these studies both FL and G-CSF were initiated on day 1 and a peak of PBMC as high as 1×10⁵/μl was observed on day 8. FL in combination with GM-CSF was much less effective. Brasel, K. et al., Blood 90:3781, 1997. Since it was previously reported that PBMC mobilized by G-CSF alone peaked on day 5 to 6, (Molineux, G. et al., Blood 75:563, 1990) we initiated G-CSF treatment on day 4 to allow for maximal synergy of both growth factors. In fact, a peak of PBMC under treatment with FL+G-CSF was detected on day 10 and an average of 1.75×10⁵ PBMC/μl was counted. This peak represents an almost 2-fold increase in PBMC numbers when compared to the experiments performed by Brasel, K. et al., Blood 90:3781, 1997. Thus, we show in this study that optimized timing of growth factor administration can further enhance the synergy between G-CSF and FL.

However, the peak of PBMC does not necessarily reflect the ideal time point for the collection of the desired cellular population from mobilized PB. Therefore, we were mainly interested in the kinetics of mobilization of FC and HSC. FC have been previously shown to be critical in engraftment of murine allogeneic HSC across MHC-barriers (Kaufman, C. L. et al., Blood, 84:2436, 1994; Gandy, K. L. and Weissman, I. L., Blood, 88:594a, 1996 (abstract); Aguila, U. L. et al., Immunol. Rev., 157:13, 1997). While in our previous studies 1,000 HSC (lineage⁻/Sca-1⁺/c-kit⁺) purified from bone marrow engrafted routinely in lethally irradiated syngeneic mice, even a 10-fold increase in HSC failed to rescue allogeneic recipients from irradiation-induced aplasia. When as few as 30,000 FC (CD8⁺/TCR⁻/CD3⁺) positively selected by cell sorting were added to 10,000 purified HSC, 100% of allogeneic recipients engrafted and none of these animals developed GVHD (Kaufman, C. L. et al., Blood, 84:2436, 1994).

When G-CSF alone was injected, the number of cells with facilitating phenotype (CD8⁺/αβTCR⁻/γδTCR⁻) in PB of those animals was not increased significantly compared to carrier-treated controls. In contrast FL as a single agent elevated (7-fold) the absolute numbers of FC over time and a peak occurred on day 9. Mobilization of FC by FL+G-CSF resulted in a highly significant synergy. Beginning on day 5 a continuous increase of FC was observed and a maximum (21-fold over controls) was reached on day 10. Interestingly, a similar pattern was observed for mobilization of HSC. When both factors were used in combination a more than 200-fold increase of HSC occurred from day 9 to 11. G-CSF alone and FL alone were less effective (17- and 36-fold, respectively). Our results in terms of HSC mobilization are in accordance with data presented by others identifying HSC/progenitor cells based on in vitro colony assays (Molineux, G. et al., Blood, 89:3998, 1997; Brasel, K. et al., Blood, 90:3781, 1997; Sudo, Y. et al., Blood, 89:3 186, 1997). In these studies a synergy of FL and G-CSF on the mobilization of BFU-E, CFU-GM, CFU-GEMM and CFU-S into PB was observed. However, different doses and timing of growth factor administration as well as different methods to assess the frequency of HSC make a direct comparison difficult.

Preliminary data from our laboratory suggests that FC may provide a tropic effect to maintain the HSC in a primitive state. While HSC alone undergo apoptosis in vitro, the addition of FC maintains the HSC in G₀. The fact that the kinetics for mobilization of FC and HSC are similar may suggest that the two are in close proximity in the hematopoietic microenvironment.

To understand the mechanism for the observed synergy of both growth factors on the mobilization of HSC and FC, we assessed the frequency of HSC (lineage⁻/sca-1⁻/c-kit⁺) and FC (CD8⁺/TCR⁻) in spleen and bone marrow under treatment with both growth factors alone or in combination. As shown previously, G-CSF mobilized both mature and progenitor cells into the periphery as indicated by declining cellularity in bone marrow (Molineux, G. et al., Blood, 75:563, 1990). The latter might be responsible for the slightly increased frequency of HSC in bone marrow observed in our G-CSF treated animals rather than proliferation of those cells. Interestingly, when FL was used as a single agent a highly significant increase in HSC (18-fold) and FC (5-fold) in bone marrow was observed on day 10. This is in accordance with results from Brasel et al. who reported 8.2-fold higher numbers of low-density lineage⁻/Sca-1⁺/c-kit⁺ HSC in bone marrow after treatment with 10 μg FL for 10 days when compared with untreated controls (Brasel, K. et al., Blood, 88:2004, 1996). In striking contrast, when we injected FL and G-CSF simultaneously the percentage of HSC showed a 4-fold expansion on day 7, but dropped to pretreatment levels on day 10, while the frequency of FC in bone marrow remained unchanged. This suggests that the proliferation of HSC and FC caused by FL in combination with the mobilizing effect of G-CSF might be responsible for the potent synergy of both growth factors to elevate HSC and FC numbers in PB.

In addition to the growth factor-mediated effects in bone marrow, there was a significant increase in cellularity and frequency of HSC/PC reported in spleens of FL-treated mice (Brasel, K. et al., Blood, 88:2004, 1996; Sudo, Y. et al., Blood, 89:3 186, 1997; Maraskovsky, E. et al., J. Exp. Med., 184:1953, 1996). We observed in our study a highly significant expansion of cells with FC phenotype and HSC in spleens of FL and FL+G-CSF treated mice. The percentage of FC in spleen of mice injected with FL alone increased from less than 1% on day 0 to 11% on day 10. This increase seems to be specific for certain cell types such as FC and HSC since the frequency of CD8⁺ T cells declined after FL administration. A similar observation was made by Brasel et al. who showed that an increase of CD8⁺/Thy-1⁻ cells in spleens of FL treated mice occurred (Brasel, K. et al., Blood, 88:2004, 1996). The same group reported later that a dramatic increase of dendritic cells was present in spleen under treatment with FL (Maraskovsky, E. et al., J. Exp. Med., 184:1953, 1996; Shurin, M. et al., Cellular Immunology, 179:174, 1997). When splenocytes from FL treated mice were depleted of T cells, B cells, NK cells and cells of erythroid lineage using iabs and complement, these cells could be divided into 5 groups based on their expression of the dendritic cell markers CDllc and CDllb. Interestingly, more than 50% of cells of population D (CD11c^{bright}/CD11b^{dim}) and E (CD11c^{bright}/CD11b⁻) coexpressed CD8 (Maraskovsky, E. et al., J. Exp. Med., 184:1953, 1996; Pulendran, B. et al., J. Immunol. 159:2222, 1997). Whether these two populations mediate a graft-facilitating effect is currently under investigation in our laboratory. However, El-Badri et al. showed recently that dendritic cells isolated from murine bone marrow did not facilitate engraftment of purified HSC across MHC-barriers (El-Badri, N. S. et al., Exp. Hematol., 26:110, 1998). Nevertheless, our preliminary data indicate that CD8⁺/TCR⁻ cells from PB and spleens of FL+G-CSF treated animals facilitate engraftment of allogeneic bone marrow across MHC-barriers (unpublished observation).

This observation is further confirmed by the superior engraftment-potential of PB from BLO.BR donors mobilized with FL and FL+G-CSF in fully ablated allogeneic B10 mice. One hundred percent and 83% of animals transplanted with 1×10⁶ FL+G-CSF and FL mobilized PBMC harvested on day 10 were rescued from irradiation-induced aplasia, respectively. The transplanted PBMC contained 0.63 or 0.64×10⁵ HSC and 2.10 or 1.21×10⁵ FC after treatment with FL+G-CSF and FL alone, respectively. In contrast, similar amounts of HSC and FC present in G-CSF mobilized PB rescued only 33% of lethally irradiated animals indicating a qualitative disadvantage of these cells after treatment with G-CSF alone. When PBMC were harvested on day 7 of growth factor administration the engraftment potential was less efficient. Therefore the collection of PB on day 10 at which the highest numbers of HSC and FC in PB were detectable seems to be favorable. Moreover, the long term repopulating potential of HSC and FC from FL and FL+G-CSF treated allogeneic donors was superior when compared to donors treated with G-CSF alone. However, the development of GVHD as a result of high numbers of T cells and NK cells in whole PB has limited the long-term survival in those animals.

In summary, treatment of mice with FL results in proliferation of cells with FC phenotype in bone marrow and spleen. In animals treated with a combination of FL and G-CSF both FC and HSC were most efficiently mobilized into PB and peak levels for both populations were detected on day 10. This strategy might be useful in the clinical setting especially when HLA-disparities between donor and recipient exist and FC are needed to achieve HSC-engraftment. However, improved collection and processing of mobilized PB to contain mainly FC and HSC yet reduce the amount of contaminating T cells and NK cells might be necessary to avoid GVHD and enhance the engraftment-potential.

### 8. EXAMPLE 3

### TNFα and GM-CSF Enriched FC and HSC ex vivo

8.1. Discussion

8.1.1. Animals

Six to eight week old B10.BR SG SNJ (H-2K^{k}) and C57BL/10SNJ (H-2K^{b}) mice were purchased from Jackson Laboratory, Bar Harbor, Me. The animals were housed in a pathogen-free animal facility at the Institute for Cellular Therapies, Allegheny University of the Health Sciences, Philadelphia, Pa., and cared for according to specific Allegheny University and National Institutes of Health animal care guidelines.

8.1.2. Treatment with 5 Fluorouracil (5 FU)

5 fluorouracil (5 FU), is commercially available as Adrucil (Pharmacia Inc., Kalamazoo, Mich.). Mice were treated with a single dose of 5 FU (150 mglkg body weight) by i/v injection into the tail vein. Each dose of 5 FU was drawn from a stock solution of 10 mg/ml in PBS. The stock bottle was stored at 4° C. Bone marrow was collected at day 5 after 5 FU administration.

8.1.3. Facilitating Cell Culture

Mice were treated with 5 FU as described above. BM cells collected from the 5 FU-treated mice are highly enriched for early hematopoietic progenitor cells. The estimated count of BM cells is 2×10⁶ to 3×10⁶ cells per animal. Tibias and femurs were aseptically removed from animals and their ends cut off. BM cells were expelled into a Petri dish by forcing complete medium (CM) [RPMI 1640 medium (Gibco BRL, Grand Island, N.Y.), 10% FBS (Gibco BRL, Grand Island, N.Y.), 2 mM L-glutamine (Gibco BRL, Grand Island, N.Y.), 5×10⁻⁵M2-mercaptoethanol (Bio-Rad Laboratories, Richmond, Calif.), 10 mM Hepes (Gibco BRL, Grand Island, N.Y.), 0.1 mM Non essential AA (Gibco BRL, Grand Island, N.Y.), 1 mM Na Pyruvate (Gibco BRL, Grand Island, N.Y.), and 50 μg/ml Gentamicin (Gibco BRL, Grand Island, N.Y.)] through the bone shaft. The BM cells were suspended, passed through a 30 μm nylon mesh (Tetko, Briarcliff Manor, N.Y.), centrifuged and the pelleted cells were depleted of red blood cells by addition of 4 ml red blood cell lysing buffer (Sigma, St. Louis, Mo.). Following a 5 min incubation at room temperature, 15 ml CM were added to stop the lysing process, and suspension was centrifuged for 10 min at 1000 rpm and 4° C. After washing, BM cells were counted, diluted to the concentration 0.25×10⁵ cells/ml, and cultured in 6-well plates (Corning Inc., Corning N.Y.) plated at 4 ml per well at 37° C. and 5% CO₂ in CM, supplemented with GM-CSF (Sigma, St. Louis, Mo.) at 1,000 U/ml. At day 7 cultured cells were collected from the plates by gentle pipetting, centrifuged, count and subcultured at the same culture regimens as for first 7 days with TNFα (Genzyme, Cambridge, Mass.) at the concentration 200 U/l instead of GM-CSF as a growth factor for one day. Harvested cells (total length of culture 8 days) were counted, and analyzed.

8.1.4. Flow Cytometry

Flow cytometry analyses of bone marrow after 5 FU-treatment, and of cultured cells were performed on a Becton Dickinson dual laser FACSCalibur. Cells were incubated with directly conjugated monoclonal ABS: anti- Class I and Class II, CD2, CD28, CD34, CD3, CD8, αβTCR, γδTCR, Thy 1, Sca-1, c-kit, CD45, CD86, CD11b, CD11c, CD4, B220, GR1, Thy1.2, NLDC/145, FAS, CD54, CD40L. All mAbs listed above were purchased from Pharmingen (San Diego, Calif.) and Becton Dickinson (San Jose, Calif.).

8.1.5. Preparation of Mixed Allogeneic Chimeras

A detailed procedure for preparation of mixed allogeneic murine chimeras has been published extensively (Ildstad et al., 1985, J. Exp. Med., 162:231; Ildstad et al., 1986, J. Immunol., 136:28; Sykes et al., 1989, J. Immunol., 143:3503; Kaufman et al., 1994, Blood, 94:2436-2446). Briefly, B10 recipient mice were exposed to 950 cGy of total body (TBI) irradiation, ablating the native chematopoiesis. Donor bone marrow inoculum was aseptically prepared as single cell suspension, T-cell depleted using RAMB polyclonal serum (prepared and titrated in the laboratory) as described (Ildstad and Sachs, 1984, Nature, 307:168; Ildstad et al., 1985, J. Exp. Med., 162:231.) and administered to the recipients within 5 hours of irradiation, via a 0.5 ml intravenous injection into the tail vein. Lethally irradiated animals received a mixture of 5×10⁶ RAMB treated B10 (syngeneic), plus 5×10⁶ RAMB treated BR (allogeneic) BM cells, plus 0.25×10⁶ cultured cells.

Animals were examined daily for evidence of infection, and GVHD. Peripheral blood lymphocytes (PBLs) and skin biopsy specimens were collected monthly to evaluate the level of donor chimerism and for microscopic evaluation of GVHD.

8.1.5.1. Characterization of Chimeras by Plow Cytometry

Recipients were characterized for engraftment with syngeneic and/or allogeneic donor hematopoiesis using flow cytometry to determine the percentage of PBLs bearing (B10) or H-2^{k} (BLOBR) markers. Peripheral blood was collected into heparinized plastic serum vials, and diluted 1:3 in M 199 media (Gibco, Gaithersburg, Md.). After thorough mixing, the cell suspension was layered over 1.5 ml of room temperature Ficoll-Paque (Pharmacia Biotech Piscataway, N.J.), centrifuged for 30 minutes at 23° C., and 400 g. The lymphocytes were collected from the media/gradient interface and washed with M 199 media. Cells were stained with directly labeled anti-H-2b and H-₂^{k} mAbs. As a negative control were used directly labeled with a same fluorochrome as anti-Class I antibodies anti-human CD3 (Lue 4) mAb. Arbitrary levels on log scale was selected based on the inflection point at which staining of the control negative population was minimized while retaining maximal numbers of positively stained cells. Flow analysis was performed on a Becton Dickinson dual laser FACSCalibur. All nAbs are purchased from Pharmingen (San Diego, Calif.) and Becton Dickinson (San Jose, Calif.).

8.1.5.2. Microscopy Evaluation of GVHD

Skin biopsy specimens were fixed in formalin and frozen in OCT compound. After 3 days of fixation in formalin, specimens were routinely processed and embedded in paraffin. Five micron H & E stained sections were used for microscopic evaluation of GVHD. Mononuclear cell infiltration and corresponding structure of damage of skin was assessed by light microscopy. In case the mononuclear cells infiltration was observed the immunohistochemistry staining of frozen biopsy specimens was performed. The mAbs against donor specific markers were used to identify the donor derived cells.

8.1.5.3. Morphologic Studies

For morphologic studies freshly isolated or generated in culture cells were incubated on poly-L-lysine-coated or silanated slides for 40 min at 37° C., washed, fixed in cold methanol and used for examination after the modified Wright-Giemsa staining, using Leukostat Stain Kit (Fisher, Pittsburgh, Pa.).

8.2. Results

A cell population with the ability to enhance the level of MHC-disparate donor chimerism in lethally ablated recipients has been generated in culture. A murine model of mixed allogeneic reconstitution with a ratio of 1:1, syngeneic to allogeneic, T-cell depleted bone marrow cells (5×10⁶ B10_{RAMB}>10) was used as a model to study the facilitating effect of cell populations generated in culture. T-cell depletion was accomplished using rabbit anti-mouse brain (RAMB) polyclonal serum which cross-reacts with mouse T-cells. Resulting chimeras have a low durable level of donor chimerism. When 250,000 cultured cells were administered in addition to RAMB-treated B10 and BR bone marrow inoculum for the recipient reconstitution donor chimerism was enhanced to over 96% in all animals (FIGS. 4A and 4B). Four percent of the cultured cells have the FC phenotype (CD8⁺/αβTCR⁻). Percentage of cells with CD8⁺/αβTCR-/B7.2⁺/CD11c⁺ phenotype which is believed to characterize the Lymphoid Dendritic cell (LDC) population was also 4% (FIGS. 5A-5D).

8.3. Discussion

Example 3 demonstrates a method to culture cells with the ability to facilitate allogeneic chimerism. The phenotypic characterization of FC has been described as Class II⁺/CD8^{dull/intermediate}/CD3⁺/αβTCR⁻/γδTCR⁻/NK⁻, and this cell population has been demonstrated to be necessary for HSC to engraft in MHC-disparate recipients (Kaufman et al., 1994, Blood, 94:2436-2446). Four percent of cells in the culture system do have the main phenotypic characteristic of FC: they are CD8⁺ and αβTCR⁻. The absolute number of cells with this phenotype was about 10,000 when 250,000 of cultured cells were added to the mixture of T-cell depleted syngeneic and allogeneic BM cells for mixed allogeneic chimera preparation. This number (10,000 of cultured CD8⁺/αβTCR⁻ cells) is a quantity comparable to 30,000 FC freshly isolated from the bone marrow in the applied model. The observed facilitating effect is likely due to the presence of FC in the cell culture, and direct evidence of which particular subset in the heterogeneous culture cell population provided the FC effect in repopulating the fully ablated allogeneic recipient in currently being sought.

### 9. EXAMPLE 4

### TNFα, GM-CSF, G-CSF, and FL Enriched FC and HSC ex vivo

A. Materials and Methods

9.0.1. Animals

Six to eight week old B10.BR SG SNJ (H-2K^{k}) and C57BL/10SNJ (H-2K^{b}) mice were purchased from Jackson Laboratory, Bar Harbor, Me. The animals were housed in a pathogen-free animal facility at the Institute for Cellular Therapies, Allegheny University of the Health Sciences, Philadelphia, Pa., and cared for according to specific Allegheny University and National Institutes of Health animal care guidelines.

9.0.2. Donor Treatment with 5 Fluorouracil (5 PU), FL, and G-SCF

5 fluorouracil (5 FU) is commercially available as Adrucil (Pharmacia Inc., Kalamazoo, Mich.). Mice from which BM was harvested were treated with a single dose of 5 FU (150 mg/kg body weight) by i/v injection into the tail vein. Each dose of 5 FU was drawn from a stock solution of 10 mg/ml in PBS. The stock bottle was stored at 4° C. Bone marrow was collected at day 5 after 5 FU administration.

Animals from which splenocytes were collected were treated with 5 FU, as above, as well as FL and G-CSF. FL was obtained from Immunex Corp. (Seattle, Wash.), and diluted with 0.9% saline. Animals were treated with 10 daily subcutaneous injections of 10 μg per day.

G-CSF (Neupogen) was obtained from Amgen, Inc. (Thousand Oaks, Calif.), and diluted with 0.9% saline, supplemented with 0.1% mouse serum albumin (Sigma, St. Louis, Mo.). Animals were treated with 7 daily subcutaneous injections of 7.5 μg/kg.

9.0.3. Facilitating Cell Culture

Mice were treated with 5 FU or 5 FU, FL, and G-CSF, as described above. The estimated count of BM cells harvested is 2×10⁶ to 3×10⁶ cells per animal. Tibias and femurs were aseptically removed from animals and their ends cut off. BM were expelled into a Petri dish by forcing complete medium (CM) [RPMI 1640 medium (Gibco BRL, Grand Island, N.Y.), 10% FBS (Gibco BRL, Grand Island, N.Y.), 2 mM L-glutamine (Gibco BRL, Grand Island, N.Y.), 5×10⁻⁵M2-mercaptoethanol (Bio-Rad Laboratories, Richmond, Calif.), 10 mM Hepes (Gibco BRL, Grand Island, N.Y.), 0.1 mM Non-essential AA (Gibco BRL, Grand Island, N.Y.), 1 mM Na Pyruvate (Gibco BRL, Grand Island, N.Y.), and 50 μg/ml Gentamicin (Gibco BRL, Grand Island, N.Y.)] through the bone shaft.

The estimated count of harvested splenocytes is around 300×10⁶ per animal. Splenocytes were obtained by grinding spleen with frosted microscope slides (Erie Scientific, Portsmouth, N.H.).

The BM cells and splenocytes were suspended, passed through a 30 μm nylon mesh (Tetko, Briarcliff Manor, N.Y.), centrifuged and the pelleted cells were depleted of red blood cells by addition of 4 ml red blood cell lysing buffer (Sigma, St. Louis, Mo.). Following a 5 min incubation at room temperature, 15 ml CM were added to stop the lysing process, and suspension was centrifuged for 10 min at 1000 rpm and 4° C.

After washing, BM cells and splenocytes were counted, diluted to the concentration 0.25×10⁶ cells/ml, and cultured in 6-well plates (Corning Inc., Corning N.Y.) plated at 4 ml per well at 37° C. and 5% CO₂ in CM. BM was cultured in the presence of GM-CSF (Sigma, St. Louis, Mo.) at 1,000 U/ml and TNFα (Genzyme, Cambridge, Mass.) at 200 U/ml for 8 days. Splenocytes were cultured with SCF (Genzyme, Cambridge, Mass.) at 2 μ/mil, FL (Immunex Corp., Seattle, Wash.) at 500 ng/ml, GM-CSF at 1000 p/ml, IL-7 (Genzyme, Cambridge, Mass.) at 10 ng/ml, IL-12 (Genzyme, Cambridge, Mass.) at 10 ng/ml and TNFα at 200 U/ml for 10 days. Harvested cells (total length of culture 8 or 10 days depending on the culture conditions, as described above) were counted, and analyzed.

9.0.4. Flow Cytometry

Flow cytometry analyses of bone marrow and spleen after donor treatment, and of cultured cells were performed on a Becton Dickinson dual laser FACSCalibur. Cells were incubated with directly conjugated monoclonal ABS: anti-Class I and Class II, CD2, CD28, CD34, CD3, CD8, αβTCR, γδTCR, Thy 1, Sca-1, c-kit, CD45, CD86, CD11b, CD11c, CD4, B220, GR1, Thy1.2, NLDC/145, FAS, CD54, CD40L. All mAbs listed above were purchased from Pharmingen (San Diego, Calif.) and Becton Dickinson (San Jose, Calif.).

9.0.5. Preparation of Mixed Allogeneic Chimeras

A detailed procedure for preparation of mixed allogeneic murine chimeras has been published extensively (Ildstad et al., 1985, J. Exp. Med., 162:231; Ildstad et al., 1986, J. Immunol., 136:28; Sykes et al., 1989, J. Immunol., 143:3503; Kaufman et al., 1994, Blood, 94:2436-2446). Briefly, B10 recipient mice were exposed to 950 cGy of total body (TBI) irradiation, ablating the native hematopoietic system. Donor bone marrow inoculum was aseptically prepared as a single cell suspension, T-cell depleted using RAMB polyclonal serum (prepared and titrated in the laboratory) as described (Ildstad and Sachs, 1984, Nature, 307:168; Ildstad et al., 1985, J. Exp. Med., 162:231.) and administered to the recipients within 5 hours of irradiation, via a 0.5 ml intravenous injection into the tail vein. Lethally irradiated animals received a mixture of 5×10⁶ RAIB treated B10 (syngeneic), plus 5×10⁶ RAIB treated BR (allogeneic) BM cells, plus 0.25×10⁶ cultured cells. Animals were examined daily for evidence of infection, and GVHD. Peripheral blood lymphocytes (PBLs) and skin biopsy specimens were collected monthly to evaluate the level of donor chimerism and for microscopic evaluation of GVHD.

9.0.5.1. Characterization of Chimeras by Flow Cytometry

Recipients were characterized for engraftment with syngeneic and/or allogeneic donor hematopoiesis using flow cytometry to determine the percentage of PBLs bearing H-2b (B10) or H-2^{k} (B10BR) markers. Peripheral blood was collected into heparinized plastic serum vials, and diluted 1:3 in M 199 media (Gibco, Gaithersburg, Md.). After thorough mixing, the cell suspension was layered over 1.5 ml of room temperature Ficoll-Paque (Pharmacia Biotech Piscataway, N.J.), centrifuged for 30 minutes at 23° C., and 400 g. The lymphocytes were collected from the media/gradient interface and washed with M 199 media. Cells were stained with directly labeled anti-H-2b and H-2^{k} mabs. As a negative control were used directly labeled with a same fluorochrome as anti-Class I antibodies anti-human CD3 (Lue 4) mAb. Arbitrary levels on log scale was selected based on the inflection point at which staining of the control negative population was minimized while retaining maximal numbers of positively stained cells. Flow analysis was performed on a Becton Dickinson dual laser FACSCalibur. All mAbs were purchased from Pharmingen (San Diego, Calif.) and Becton Dickinson, (San Jose, Calif.).

9.0.5.2. Microscopy Evaluation of GVHD

Skin biopsy specimens were fixed in formalin and frozen in OCT compound. After 3 days of fixation, specimens were routinely processed and embedded in paraffin. Five micron H & E stained sections were used for microscopic evaluation of GVHD. Mononuclear cell infiltration and corresponding structure of damage of skin was assessed by light microscopy. In case the mononuclear cells infiltration was observed the immunohistochemistry staining of frozen biopsy specimens was performed. The mAbs against donor specific markers were used to identify the donor derived cells.

9.0.5.3. Morphologic Studies

For morphologic studies freshly isolated or generated in culture cells were incubated on poly-L-lysine-coated or silanated slides for 40 min at 37° C., washed, fixed in cold methanol and used for examination after the modified Wright-Giemsa staining, using Leukostat Stain Kit (Fisher, Pittsburgh, Pa.).

B. Results

A cell population with the ability to enhance the level of MHC-disparate donor chimerism in lethally ablated recipients has been generated in culture. A murine model of mixed allogeneic reconstitution with a ratio of 1:1, syngeneic to allogeneic, T-cell depleted bone marrow cells (5×10⁶ B10_{RAMB}≧B10) was used as a model to study the facilitating effect of cell populations generated in culture. T-cell depletion was accomplished using rabbit anti-mouse brain (RAMB) polyclonal serum which cross-reacts with mouse T-cells. Resulting chimeras have a low but durable level of donor chimerism. When 250,000 cultured cells were administered in addition to RAMB-treated B10 and BR bone marrow inoculum for the recipient reconstitution donor chimerism was enhanced to over 75% in all animals.

C. Discussion

Example 4 demonstrates a method to culture cells with the ability to facilitate allogeneic chimerism without causing GVHD. The phenotypic characterization of FC has been described as Class II⁺/CD8^{dull/intermediate}/CD3⁺/αβTCR⁻/γδTCR⁻/NK⁻, and this cell population has been demonstrated to be necessary for HSC to engraft in MHC-disparate recipients (Kaufman et al., 1994, Blood, 94:2436-2446). A murine model of mixed allogeneic reconstitution with a ratio of 1:1, syngeneic to allogeneic, T-cell depleted bone marrow cells (5×10⁶ B10_{RAMB}+5×10⁶ BR_{RAMB}>B10) was used as a model for evaluation of cultured cells to enhance the allogeneic chimerism. T-cell depletion was accomplished using rabbit anti-mouse brain (RAMB) polyclonal serum which cross-reacts with mouse T-cells. Resulting chimeras has a low level of donor chimerism.

The administration of 0.25×10⁶ cultured cells in addition to RAMB-treated B10 and BR bone marrow inoculum for the recipient reconstitution enhanced donor chimerism to over 75% in all animals. Donor chimerism was durable, and no signs of GVHD developed during the 6 month observation period. The observed facilitating effect is likely due to the presence of FC in the cell culture, and direct evidence of which particular subset in the heterogeneous culture cell population provided the FC effect in repopulating the fully ablated allogeneic recipient is currently being sought.

The yield of the FC cultured in the described model is such that one BM culture donor is sufficient for allogeneic reconstitution of one recipient, and one splenocyte culture system donor for 50 recipients.

Four and six percent of cells in BM and spleen culture systems respectively have the main phenotypic characteristic of FC: CD8⁺ and αβTCR⁻. The flow cytometry analysis of cultured cells provides interesting data concerning the lineage of origin of FC. All cells with FC markers are positive for Lymphoid dendritic cell (LDC) markers as well. We can suggest that cells with the described phenotype (CD8⁺/αβTCR⁻/B7.2⁺/CD11c⁺) are the same cells that promote the FC function in chimeric animals. Even though unmodified heterogeneous populations of cultured cells were used for chimera preparation, there were no cells with the phenotype CD8⁺, αβTCR⁻, but B7.2⁻, and CD11c⁻. Further studies are required to explore the hypothesis that FC belong to the LDC hematopoietic cell compartment, and to purify FC from the cultured cell populations.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated herein by reference for all purposes.

What is claimed is:

1. A cellular composition comprising mammalian peripheral blood mononuclear cells enriched in hematopoietic stem cells and facilitating cells, and depleted of graft-versus-host disease producing cells.

2. A cellular composition comprising human peripheral blood mononuclear cells enriched in hematopoietic stem cells and facilitating cells.

3. The cellular composition of claim 2 which is further depleted of graft-versus-host disease producing cells.

4. A cellular composition of claim 1 comprising mammalian peripheral blood mononuclear cells enriched in CD34⁺ and CD8⁺ cells, and depleted of αβTCR⁺ and γδTCR⁺ cells.

5. A cellular composition of claim 3 comprising human peripheral blood mononuclear cells enriched in CD34⁺ and CD8⁺ cells, and depleted of αβTCR⁺ and γδTCR⁺ cells.

6. A pharmaceutical composition for reconstitution of bone marrow of a recipient, comprising the cellular composition of claim 2, 3, or 5 in which the hematopoietic stem cells and facilitating cells are histocompatible with the recipient.

7. A method for preparing mammalian peripheral blood mononuclear cells enriched in hematopoietic stem cells and facilitating cells, comprising:
(a) treating a donor with a composition that activates FLT3 and the G-CSF receptor, so that hematopoietic stem cells and facilitating cells are mobilized into the circulation;
(b) collecting peripheral blood mononuclear cells from the donor when both hematopoietic stem cells and facilitating cells are mobilized; and
(c) depleting the collected peripheral blood mononuclear cells of graft-versus-host disease producing cells.

8. A method for preparing human peripheral blood mononuclear cells enriched in hematopoietic stem cells and facilitating cells, comprising:
(a) treating a human donor with a composition that activates FLT3 and the G-CSF receptor, so that hematopoietic stem cells and facilitating cells are mobilized into the circulation; and
(b) collecting peripheral blood mononuclear cells from the donor when both hematopoietic stem cells and facilitating cells are mobilized.

9. The method of claim 8 further comprising depleting the collected peripheral blood mononuclear cells of graft-versus-host disease producing cells.

10. The method of claim 8 or 9 in which the hematopoietic stem cells and facilitating cells are mobilized into the circulation by treating the donor with G-CSF and FLT-3 ligand.

11. The method of claim 10 in which the donor is treated daily and the peripheral blood mononuclear cells are collected from the donor at least 8 days subsequent to the initial treatment.

12. A method for reconstituting bone marrow in a human recipient, comprising administering the pharmaceutical composition of claim 6 into the recipient.

13. A method for preparing mammalian hematopoietic cells enriched in hematopoietic stem cells and facilitating cells comprising treating a mammalian cell population with a composition that activates the GM-CSF receptor and the TNF receptor so that hematopoietic stem cells and facilitating cells are increased in number.

14. A method for preparing human hematopoietic cells enriched in hematopoietic stem cells and facilitating cells comprising treating a human cell population with a composition that activates the GM-CSF receptor and the TNF receptor so that hematopoietic stem cells and facilitating cells are increased in number.

15. The method of claim 13 or 14 further comprising treating the cell population with a composition that activates FLT3, the SCF receptor, the G-CSF receptor, the IL-7 receptor, the IL-12 receptor, or any combination thereof.
